(19) United States
(12) Patent Application Publication
(10) Pub. No.: US 2024/0228492 A1
(43) Pub. Date: Jul. 11, 2024

(54) Process For Purification of Chemical Components From Plant Matter

(71) Applicant: KB Crash Creations LLC, West Wyoming, PA (US)

(72) Inventor: Cory Kilheeney, Dallas, PA (US)

(73) Assignee: KB Crash Creations LLC, West Wyoming, PA (US)

(21) Appl. No.: 18/406,981

(22) Filed: Jan. 8, 2024

Related U.S. Application Data
(60) Provisional application No. 63/479,119, filed on Jan. 9, 2023.

(51) Int. Cl. C07D 471/14; B01D 9/00; B01D 11/02

(52) U.S. Cl. CPC C07D 471/14; B01D 9/0036; B01D 9/0081; B01D 11/0288; B01D 11/0292; B01D 2009/0086

(57) ABSTRACT

The present invention relates to a three-phase methodology to obtain a highly concentrated, purified chemical component from plant material in an industrial setting. The three phases of the methodology of the present invention include Phase 1: Preparing and solubilization, Phase 2: Separation and Salting and Phase 3: crystallization. The methodology results in dried composition purified from a plant material having at least 55%, 60%, 65%, 70%, 75%, 80%, 85%, 90%, or 95% of the chemical component by weight.

### BACKGROUND OF THE INVENTION

It is known that extracts and chemical components from plant materials have medicinal and beneficial purposes. However, although certain quantities have successfully been extracted for teas, supplements, pharmaceuticals, and the like, purifying such components for these commercial purposes has generally been challenging for a variety of reasons.

In particular, purifying alkaloids and similar chemical components from plant material in high concentrations and in large quantities has generally been difficult. Many have tried to do so but often with little success. Prior to the present invention, extraction techniques often resulted in a purity of the alkaloid from about 1-45% by weight.

Accordingly, a need exists to purify chemical components from plant material in higher concentrations, as compared to traditional methods. Additionally, a need exists to do so in large quantities. Finally, yet another need exists to efficiently and effectively purify and extract chemical components that have medicinal and beneficial properties for incorporation into a variety of applications. An additional need exists for performing such a purification with “green” solvents, bases, and acids.

### SUMMARY OF THE INVENTION

The present invention relates to methods for purifying one or more chemical components from a starting plant material e.g., on a large or industrial scale. The starting plant material has a weight greater than 100 kilograms, e.g., ranging from about 100 kilograms to about 1000 kilograms (e.g., about 100, 150, 200, 250, 300, 350, 400, 450, 500, 550, 600, 650, 700, 750, 800, 850, 900, 950, or 1000 kilograms), wherein the starting plant material has about 1% to about 2% (e.g., about 1%, 1.25%, 1.5%, 1.75%, 2%) by weight of the chemical components (e.g., alkaloids) to be isolated and purified. The inventive methods include the steps of:
a. solubilizing one or more alkaloids from starting plant material in a solubilizer (e.g., an acid or base) to thereby obtain solubilized plant material;
b. mixing or mashing the solubilized plant material within a solvent, to thereby obtain a mixture having non-soluble plant material, one or more solubilized chemical components, and the solvent;
c. mechanically separating most or all of the non-soluble plant material in the mixture from the one or more solubilized chemical components and the solvent to thereby obtain a mixture having optionally a portion of non-soluble plant material, one or more solubilized chemical components, and the solvent;
d. if a portion of the non-soluble plant material exists, then optionally filtering the mixture (e.g., one or more times with a series of decreasing filter pore sizes) having the portion of non-soluble plant material, one or more solubilized chemical components and the solvent to further remove any residual non-soluble plant material; (the result from step c) and/or step d) is a mechanically separated solution having the one or more solubilized chemical components and the solvent)
e. salting the mixture having one or more solubilized chemical components and solvent with a salting agent (e.g., polarity changer or proton donor), to thereby obtain a salted, solution having the salting agent, one or more chemical components and the solvent;
f. separating a portion (e.g., most or all) of the solvent from the one or more solubilized chemical components to obtain a salted solution having the salting agent, one or more solubilized chemical components and optionally a residual portion of the solvent;
g. assessing the purity of the chemical components (e.g., by measuring the refractive index and/or pH) of the salted solution having the salting agent, one or more solubilized chemical components having optionally a portion of the solvent, wherein the assessment (e.g., refractive index or pH) meets a criteria (e.g., refractive index of greater than about 22 and/or pH greater than 3.5);
h. separating the residual portion of the solvent, if any, from the one or more chemical components in the salted solution to thereby obtain a salted solution having the salting agent and one or more chemical components;
i. crystalizing the salted solution to thereby obtain a crystalline solid having the one or more chemical components and the salting agent;
j. separating the salting agent (e.g., polarity changer or proton donor) from the crystalline solid having the one or more chemical components to thereby obtain a purified crystalline solid having the one or more chemical components; and
k. dehydrating the purified crystalline solid having the one or more chemical components into a powder to obtain a dried composition having at least 55% of the chemical component by weight.

The amount of purified crystalline solid product having the one or more chemical components ranges from about 1% to about 2% (e.g., about 1%, 1.25%, 1.5%, 1.75%, 2%) by weight of the raw starting material. The starting plant material has about 1% to about 2% by weight of the chemical components. As such, most or all of the chemical components present in the starting plant material is recovered by the inventive methodology and present in the final product on an industrial/commercial scale. Hence, in certain embodiments, the methodology described herein provides a product wherein about 90% to about 100% (e.g., about 90, 91, 92, 93, 94, 95, 96, 97, 98, 99, 100%) of the amount of the chemical component is recovered from that in the starting raw plant material. The methods of the present invention result in an efficient highly purified product. The ratio of the weight of the starting plant material to the weight of the of the chemical component in the purified crystalline product ranges between about 100:1 to about 100:2. In an aspect, the product has an amount of the chemical component present in a range from about 55% to about 100% (e.g., at least 55%, 60%, 65%, 70%, 75%, 80%, 85%, 90%, 95%, 96%, 97%, 98%, 99%, or 100) by weight.

In an embodiment, the starting plant material can be optionally pretreated with an acid to remove fatty acids and/or a solvent to remove certain components such as chlorophyll and non-structural carbohydrates. Examples of fatty acids include chlorophyll, saponins, flavonoids, tannins, terpenes, iridoids, monoterpenoids, triterpenoids, phenolic compounds, and glucosinates. These can be summarized as secondary metabolites. Carbohydrates that can be removed include, for example, cellulose, oligosaccarhides, polysaccharides, glucose, fructose, mannose, galactose, xylose, arabinose, xylan, glucuronoxylan, arabinoxylan, glucomannan, xyloglucan, mannan, glucans, pectin, plant gums, fructans, galactose, ribose, maltose, lactose, raffinose, fructooligosaccharide, manaoligosaccharide, inulin, and starch.

The plant material used in the methodology includes e.g., bark, pollen, root system, root stem, shoot system, stem, leaf, flower, and fruit. The starting plant material for the present invention can be derived from any plants with medicinal or beneficial properties and examples are further described herein.

In an embodiment, the solubilizer that can be used for the methodology can be any base e.g., a mineral alkaline material. Bases for use in the present invention include calcium hydroxide, sodium carbonate, sodium hydroxide, potassium hydroxide, potassium carbonate, calcium carbonate or a combination thereof. Similarly, acids that can be used as a solubilizers in the present invention include glacial acetic acid, hydrochloric acid, carbonic acid, sulfuric acid, nitric acid, sulfuric acid, hydrofluoric acid, citric acid, phosphoric acid, ascorbic acid, or a combination thereof. The present invention can utilize any Lewis acid or base. A Lewis acid refers to a chemical species that contains an empty orbital which is capable of accepting an electron pair from a Lewis base to form a Lewis adduct. A Lewis base, then, is any species that has a filled orbital containing an electron pair which is not involved in bonding but may form a covalent bond with a Lewis acid to form a Lewis adduct. Such acids and bases are known in the art or later developed and can be used with the methodology of the present invention.

The solvent used in the methods described herein can be any non-polar solvent. Solvents of the present invention include those with a dielectric constant below about 5. Solvents for use with the present invention include, for example, limonene, p-cymene, pinene, cyclopentyl ethyl ether, ethyl lactate, 2-methyloxolaine, glycerol, Isopopanol, n-propylacetate, i-propylacetate, 1-butanol, 2-butanol, sulpholane, anisole, ethylene, propylene carbonate, dimethyl carbonate, tert amyl methyl ether, methyl laurate, 2-methyltetrahydrofuran, 2,2,5,5-tetramethyloxolane, cetyltrimethylammonium chloride, dimethyldodecylamine oxide, sodium dodecyl sulfate, dimethoxyethane, methylene chloride, polyethylene glycol, ethanol, methanol, petroleum ether, n-hexane, chloroform, methyl chloride, ethyl acetate, hexane, toluene, naptha, xylene, or combinations thereof.

Mechanically separating the mixture includes any technique that separates away the non-soluble plant material from the mixture. Such techniques include e.g., gravity separation, force separation, centrifuge separation, or a combination thereof.

The method of the present invention uses a salting agent (e.g., polarity changer, proton donor, Lewis acid or base), and can include, for example, glacial acetic acid, hydrochloric acid, sulfuric acid, nitric acid, sulfuric acid, hydrofluoric acid, carbonic acid, citric acid, phosphoric acid, ascorbic acid, calcium hydroxide, sodium carbonate, sodium hydroxide, potassium hydroxide, potassium carbonate or a combination thereof.

The method involves separating most or all of the solvent from the using phase separation e.g., performed in a conical fermenter.

The crystalizing step of the methodology can, in an aspect, use seed crystals along with heating, sonicating or both the salted solution to thereby obtain a crystalline solid having one or more chemical components.

The method of present invention further includes separating the salting agent (e.g., polarity changer or proton donor (e.g., acid)) from the crystalline solid having the one or more chemical components by performing one or more washes with a polar solvent. The method then includes dehydrating the purified crystalline solid into a powder comprises subjecting the purified crystalline solid having the one or more chemical components into a powder to a dehydrator or vacuum based oven at a temperature for a time period.

The resulting dried composition of the present invention has an amount ranging from 55% to 100% (e.g., at least 55%, 60%, 65%, 70%, 75%, 80%, 85%, 90%, 91%, 92%, 93%, 94%, 95%, 96%, 97%, 98%, 99%, 100%) of the chemical component by weight. Accordingly, the present invention includes the product made by the methodology described herein.

In yet another embodiment, the inventive methodology for purifying one or more chemical components from a starting plant material; includes the following steps:
a. solubilizing the one or more chemical components (e.g., plant alkaloids) from the starting plant material in a solubilizer (e.g., an acid or base), and a solvent, to thereby obtain a mixture having non-soluble plant material, one or more solubilized chemical components, and the solvent;
b. separating non-soluble plant material in the mixture from the one or more solubilized chemical components and the solvent; to obtain a separated (e.g., mechanically separated) solution having one or more solubilized chemical components and the solvent;
c. salting the mixture having one or more solubilized chemical components with a salting agent (e.g., polarity changer or proton donor) and separating at least a portion (e.g., most or all) of the solvent from the one or more solubilized chemical components, to thereby obtain a salted solution having the salting agent, one or more chemical components and optionally a residual portion of the solvent;
d. optionally separating the residual portion of the solvent, if any, from the salted solution having the salting agent and one or more chemical components;
e. crystalizing the salted solution having the salting agent and one or more chemical components, to thereby obtain a crystalline solid having the one or more chemical components;
f. separating the salting agent and optionally any residual agent that may be present (e.g., acid, base, polarity changer or proton donor) from the crystalline solid having the one or more chemical components to thereby obtain a purified crystalline solid having the one or more chemical components; and
g. dehydrating the purified crystalline solid having the one or more chemical components into a powder to obtain a crystalline product/dried composition having at least 55% of the chemical component by weight.

The purification methodology of the present invention advantageously allows for extracted plant-derived chemical components in high concentrations and processed in large quantities. The inventive methodology results in alkaloid products having greater than 55% alkaloid by weight or greater.

### BRIEF DESCRIPTION OF THE DRAWINGS

The foregoing and other objects, features and advantages of the invention will be apparent from the following more particular description of preferred embodiments of the invention, as illustrated in the accompanying drawings.

FIG. 1 is a schematic showing the steps of Phase 1 of Methodology 100: Preparing and Solubilization.

FIGS. 2A and 2B are schematics showing the steps of Phase 2 of Methodology 100: Separation and Salting.

FIGS. 3A and 3B are schematics showing the steps of Phase 3 of Methodology 100: Crystallization Phase.

FIGS. 4A-4E are schematics showing the steps of application of all three phases of Methodology 100 using Mitragyna speciosa, in powdered form referred to as Kratom as the starting plant material to obtain a product of at least about 55% mitragynine by weight.

### DETAILED DESCRIPTION OF THE INVENTION

A description of preferred embodiments of the invention follows.

The present invention relates to a methodology for purifying chemical components (e.g., alkaloids) from plant material. The methodology involves at least three phases: Phase 1: Preparing the plant material and solubilization; Phase 2: Separation from plant material from solubilized chemical components and Salting; and Phase 3: Crystallization of the purified chemical component.

### Phase 1: Preparing and Solubilization

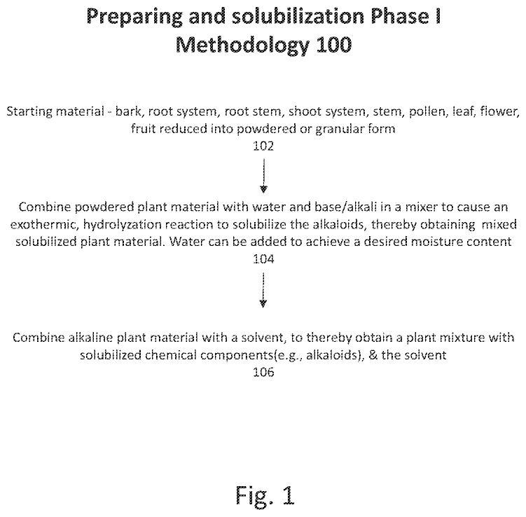

Referring to FIG. 1, Phase 1 is described. Phase 1 allows for the plant material to release chemical components including alkaloids by solubilization. The starting material can be any plant material or plant species. This methodology can be used with plant material or plant species, including genetically modified plant species. The plant material that can be used for the present invention includes, for example, bark, root system, pollen, root stem, shoot system, stem, leaf, flower, fruit or combination thereof. The methods of the present invention, in an embodiment, utilize a powdered or granular form of the plant material. Powdered forms of plant material can be made from drying, grinding of plant material and the like. Powdered forms of plant material are commercially available from, e.g., Valora Group (Florida, USA). Any plant having a chemical component that has medicinal properties and/or alkaloids or estragoles can be used. In an embodiment, the present invention utilizes kratom powder obtained from the leaves of an evergreen tree, referred to as Mitragyna speciosa.

The starting plant material has a weight ranging from about 100 kilograms to about 1000 kilograms (e.g., about 100, 150, 200, 250, 300, 350, 400, 450, 500, 550, 600, 650, 700, 750, 800, 850, 900, 950, or 1000 kilograms), wherein the starting plant material has about 10 to about 2% (e.g., about 1%, 1.25%, 1.5%, 1.75%, 2%) by weight of the chemical components (e.g., alkaloids) to be isolated and purified.

Any plant material from any plant species can be used in this methodology. Examples of plant species that can be used with this purification methodology of the present invention include, but are not limited to, Mitragyna speciosa (Kratom (leaf)), Cannabis sativa, Piper methysticum, (kava root), Hypericum perforatum (St. johns wart), Lepidium meyenii (maca), Panax quinquefolius L. (ginseng), Nymphaea caerulea blue lotus. Common examples include Ashwagandha plant, Allium sativum, Vitex negundo, stinging nettle, Orange flowers, Chamomile flowers, aloe vera, lemon grass, fenugreek, rosemary, peppermint, thyme, lavender, tulsi, ginger, and curry leaves. Additionally, plants listed in Table 1 below are more examples of those that can be used in this methodology.

TABLE 1
Plant Species Examples With Medical Properties for use with present invention

Acer saccharinum (Maple), Argemone Mexicana (Argemone - Prickly Poppy), Chionanthus virginicus
(Fringe tree), Abies balsamea (L.)(Mill., Balsam Fir), Acacia senegal (Acacia), Achillea millefolium
(Yarrow), Acorus calamus (Calamus root), Adiantum capillus-veneris (Maidenhair Fern), Aesculus
hippocastanum (Horse Chestnut), Agaricus blazei (mushroom), Agathosma betulina (Buchu), Agave
tequilana (Agave syrup), Agrimonia eupatoria L. (Agrimony), Agropyron repens (Couch grass), Alchemilla
vulgaris (Lady's Mantle) Aletris farinosa (Unicorn Root - Colic Root), Aleurites moluccanus (Kukui Nut
Oil), Allium sativum (Garlic Aloe vera, Aloe Vera Gel), Aloysia citriodora (Lemon Verbena), Alpinia
officinarum (Galangal Root), Althaea officinalis L. (Marsh Mallow Root), Anacardium occidentale (Cashew
Nut), Ananas comosus (Pineapple), Andrographis paniculata (Andrographis), Anethum graveolens (Dill),
Angelica archangelica (Angelica root), Angelica sinensis (Dong Quai), anthriscus cerefolium (Chervil),
Apium graveolens (Celery Seed), Aralia racemosa (Spikenard), Arctium lappa (Burdock root),
Arctostaphylos uva-ursi (Uva Ursi), Argania spinosa (Argan Oil), Aristolochia spp (Snakeroot), Armoracia
rusticana (Horseradish root), Arnica montana (Arnica), Artemisia absinthium L. (Wormwood), Artemisia
dracunculus (Tarragon), Artemisia vulgaris (Mugwort), Arthrospira platensis (Spirulina), Asarum canadense
(Wild Ginger), Asclepias syriaca or A. cornute (Milkweed), Asclepias tuberosa (Pleurisy Root), Aspalathus
linearis (Rooibos), Asparagus spp (Asparagus), astragalus membranaceus (Astragalus root), Atropa
belladonna (Belladonna), Avena sativa L. (Oats), Azadirachta Indica (Neem), Bacopa monnieri, Bacopa,
Berberis vulgaris L., Barberry, Betula alba, Birch bark & leaf, Bixa orellana, Annatto Seed, Borago
officinalis, Borage Seed Oil, Boswellia thurifera, Boswellia (Frankincense), Brassica napus, Canola oil
rapeseed, Brassica nigra, Mustard, Brosimum alicastrum, Ramon Nut, Bupleurum chinense, Bupleurum,
Butyrospermum parkii, Shea Nut Butter, Calendula officinalis, Calendula, Calophyllum inophyllum, Tamanu
Oil, Camellia sinensis, Tea, Camellia sinensis, Oolong Tea, Cananga adorata, Ylang-Ylang, Cannabis
sativa, Hemp Oil, Capsella bursa-pastoris, Shepherd's Purse, Capsicum minimum, Cayenne Pepper,
Caralluma fimbriata, Caralluma, Carapa guianensis, Andiroba Oil, Carica papaya, Papaya, Carthamus
tinctorius, Safflower Oil, Carum carvi, Caraway Seed, Cassia nomame, Cassia Nomame, Caulophyllum
thalictroides, Blue Cohosh Root, Ceanothus americanus, Red Root, Cedrus atlantica, Cedarwood, Atlas,
Centaurea cyanus, Cornflowers, Centaurium erythraea, Centaury, Centella asiatica (L.), Gotu Kola,
Ceratonia siliqua, Carob, Chamaelirium luteum, False Unicorn Root, Chelidonium majus, Celandine,
Chenopodium ambrosioides, pazote, Chrysanthemum leucanthemum, Oxeye Daisy, Chrysanthemum
morifolium, C. sinense, Chrysanthemum, Cichorium intybus, Chicory root, Cimicifuga racemosa, Black
Cohosh root, Cinchona spp, Peruvian Bark, Cinnamomum camphora, Camphor, Cinnamomum zeylanicum,
C. cassia, Cinnamon, Citrus aurantifolia, Lime Oil, Citrus aurantium subsp. amara or Bigaradia, Neroli,
Citrus bergamia, Bergamot Oil, Citrus brigarade, Petitgrain Oil, Citrus limonum, Lemon, Citrus paradisi,
Grapefruit, Citrus sinensis, Citrus spp., Orange, Citrus × aurantium subsp. amara, Bitter Orange, Cnicus
benedictus, Blessed Thistle, Coccinia grandis, Ivy gourd, Cocos nucifera, Coconut oil, Codonopsis pilosula,
Codonopsis, Coffea arabica, Coffee, Cola acuminata, Kola Nut, Commiphora myrrha, Myrrh, Convolvulus
Jalapa, Jalap, Copaifera Officinalis, Copaiba Balsam, Coptis spp, Coptis, Coriandrum sativum, Cilantro,
Coriandrum sativum, Coriander, Corydalis spp, Corydalis, Corylus avellana, Hazelnut Oil, Crataegus
monogyna, Hawthorn Berry, Crocus sativus, Saffron, Cucurbita pepo, Pumpkin Seed, Cuminum cyminum,
Cumin, Cupressus sempervirens, Cypress Oil, , Curcuma longa L., Turmeric, Cyclopia spp, HoneyBush,
Cymopogon citratus, C. flexuosus, Lemongrass, Cynara scolymus, Artichoke, Globe, Datura spp, Datura,
Daucus carota L., Carrot, Wild, Digitalis purpurea, foxglove digitalis, Dioscorea villosa, Wild Yam Root,
Echinacea angustifolia, Echinacea, Elettaria cardamomum, Cardamom, Eleutherococcus senticosus,
Eleuthero Root, Epilobium angustifolium, Fireweed, Epimedium grandiflorum, Epimedium, Equisetum
arvense, Horsetail, Erythroxylum catuaba, Catuaba, Erythroxylum coca, Coca, Eschscholzia californica,
California Poppy, Eucalyptus globulus, Eucalyptus, Eugenia Jambolana, Jambul, Eupatorium perfoliatum,
Boneset, Eupatorium purpureum, Joe-pye weed, Euphrasia, various species, Eyebright, Euterpe oleracea,
Acai Berry, Ferula asafoetida, Asafoetida root, Filipendula ulmaria, Meadowsweet, Foeniculum vulgare,
Fennel Seed, Forsythia suspensa, Forsythia Fruit, Fucus vesiculosis, Kelp/Bladderwrack, Fumaria
officinalis, Fumitory, Galega officinalis, Goat's Rue, Galium aparine, Cleavers, Galium odoratum, Sweet
Woodruff, Ganoderma lucidum, Reishi Mushroom, Garcinia cambogia, Garcinia Fruit, Gaultheria
procumbens, Wintergreen Oil, Gentiana lutea, Gentian, Ginkgo biloba L., Ginkgo biloba, Glechoma
hederacea L, Ground Ivy, Glycyrrhiza glabra, Licorice Root, Grifola frondosa, Maitake mushroom,
Grindelia spp, Grindelia, Gymnema sylvestre, Gurmar, Gynostemma pentaphyllum, Jiaogulan, Hamamelis
virginiana, Witch Hazel, Harpagophytum procumbens, Devil's Claw, Helianthemum canadense (L.) Michx.,
Rock Rose, Helianthus annuus, Sunflower, Helichrysum angustifolium, Immortelle Oil, Hepatica nobilis var.
obtusa, Hepatica Liver-Leaf, Hibiscus sabdariffa, Hibiscus, Hippophae rhamnoides, Sea Buckthorn Oil,
Hoodia Gordonii, Hoodia, Hordeum vulgare, Barley Grass, Humulus Lupulus, Hops, Hyacinthus
nonscriptus, Wild Hyacinth, Hydrastis canadensis, Goldenseal, Hyoscyamus niger, Henbane, Hypericum
perforatum, St. John's Wort, Hyssopus officinalis, Hyssop, Ilex paraguariensis, Yerba Mate, Impatiens
capensis, Jewel Weed, Inonotus obliquus, Chaga Mushroom, Inula helenium, Elecampane, Iris germanica,
Orris Root, Jasminum officinale, Jasmine Flower Oil, Juglans cinerea, Butternut, Juglans nigra, Black
Walnut, Juniperus communis, Juniper Berries, Lactuca virosa, Wild lettuce, Larrea tridentata, Chaparral,
Laurus nobilis, Bay Laurel, Lavandula spp, Lavender, Lawsonia inermis, Henna, Leonurus caridica L.,
Motherwort, Lepidium peruvianum, Maca root, Levisticum officinale, Lovage, Liatris odoratissima, Deer's
Tongue, Linum usitatissimum L., Flax seed, Lippia graveolens, Mexican Oregano, Lobelia inflata, Lobelia,
Lonicera periclymenum, Honeysuckle, Lycium barbarum, Goji Berries, Lycopodium clavatum,, Club Moss,
Lycopus spp., Bugleweed, Magnolia Glauca, Magnolia, Mahonia aquifolium, Oregon Grape root, Malus
domestica, Apple, Maranta arundinacea, Arrowroot powder, Marrubium vulgare L., Horehound, Matricaria
recutita, Chamomile, Medicago sativa L., Alfalfa, Melaleuca alternifolia, Tea Tree oil, Melaleuca
leucadendron, M. leucadendra, Cajeput Oil, Melissa officinalis, Lemon Balm, Mentha piperita, Peppermint,
Mentha pulegium, PennyRoyal, Mentha spicata, Spearmint, Menyanthes trifoliata, Bogbean, Momordica
charantia, Bitter Melon, Monarda didyma, Bee Balm, Wild Bergamot, Morinda citrifolia, Noni, Myrica
cerifera, Bayberry, Myristica fragrans, Nutmeg, Myroxylon pereirae, Balsam of Peru, Myrtus communis,
Myrtle, Nardostachys jatamansi, Nard Oil, Nasturtium officinale, Watercress, Nelumbo nucifera, Lotus,
Nepeta cataria, Catnip, Nigella sativa, Black Seed oil, Ocimum basilicum, Basil, Ocimum sanctum, Holy
Basil, Oenothera biennis, Evening Primrose Oil, Olea europea, Olive Oil, Opuntia ficus-indica, Prickly Pear,
Origanum majorana, Marjoram, Sweet, Origanum vulgare, Oregano, Paeonia albiflora, White Peony root,
Panax ginseng, Ginseng root, Panax quinquefolius, Ginseng Root, American, Papaver rhoeas, Red Poppy
Flowers, Passiflora incarnata, Passion Flower, Paullinia Cupana, Guarana, Pausinystalia johimbe, Yohimbe,
Pelargonium graveolens, Geranium, Persea americana, Avocado, Petasites vulgaris, Butterbur, Petroselinum
crispum, Parsley, Peumus boldus Molina, Boldo, Pfaffia paniculata, Suma Root, Phaseolus vulgaris, White
Kidney Bean, Phyllanthus emblica, Amalaki, amla, Phyllanthus niruri, Chanca Piedra, Phytolacca americana,
Poke Root, Picea mariana, Spruce Oil, Pimenta officinalis, Allspice, Pimenta racemosa, Bay Rum Oil,
Pimpinella anisum, Anise Seed, Pinus sylvestris, Pine, Piper cubeba, Cubeb, Piper longum, Long Pepper,
Pippali, Piper methysticum, Kava-Kava, Piper nigrum, Black Pepper, Piscidia piscipula, Jamaican Dogwood,
Plantago major, lanceolata L., Plantain, Plantago psyllium, Plantago ovata, Psyllium, Plectranthus barbatus,
Coleus Forskohlii, Podophyllum peltatum, May Apple, Pogostemon patchouli, Patchouli, Polygonatum
biflorum, Solomon's Seal, Polygonum multiflorum, Fo-Ti Root, Populus spp, Balm of Gilead, Prunella
vulgaris L., Self-Heal, Prunus africanum, Pygeum, Prunus armeniaca, Apricot Oil, Prunus dulcis, Almond
Oil, Prunus serotina, Wild Cherry Bark, Ptychopetalum olacoides, Muira puama, Pueraria lobata, Kudzu,
Pulsatilla nuttalliana, Pasque Flower, Punica granatum, Pomegranate, Quassia amara, Quassia, Quercus
robur, Oak Bark, Ravensara aromatica, Ravensara Oil, Rehmannia glutinosa, Rehmannia root, Rhamnus
frangula, Alder Buckthorn, Rhamnus purshiana DC, Cascara Sagrada, Rheum palmatum, Turkey Rhubarb,
Rhodiola rosea, Rhodiola, Ricinus communis, Castor Oil, Rosa spp, Rose, Rosmarinus officinalis, Rosemary,
Rubus idaeus, Raspberry Leaf, Rumex acetosella var. vulgaris, Sheep Sorrel, Rumex crispus, Yellow Dock,
Ruscus aculeatus, Butcher's Broom, Ruta graveolens L., Rue, Salix Alba, White Willow Bark, Salvia
officinalis, Sage, Salvia sclarea, Clary Sage, Sambucus nigra, Elderberry, Sanguinaria canadensis L,
Bloodroot, Sanguisorba minor, Salad Burnet, Santalum album, Sandalwood Oil, Saponaria officinalis,
Soapwort, Sassafras albidum, Sassafras root, Satureja montana, Savory, Winter, Summer, Sceletium
tortuosum, Sceletium, Schisandra chinensis, Schisandra, Scrophularia nodosa, Figwort, Scutellaria
lateriflora, Skullcap, Senna alexandrina Mill., Senna, Serenoa serrulata, Saw Palmetto, Sesamum indicum,
Sesame, Silybum marianum L., Milk Thistle Seed, Simmondsia chinensis, Jojoba Oil, Smallanthus
sonchifolius, Yacon Root, Smilax sarsaparilla, Sarsaparilla Root, Solidago virgaurea, Goldenrod, Spilanthes
acmella, Spilanthes, Stachys officinalis, Wood Betony, Stellaria media, Chickweed, Stevia rebaudiana
(Bertoni), Stevia, Styrax benzoin, Benzoin Resin, Symphytum officinale, Comfrey Leaf and root, Syzygium
aromaticum, Clove Oil, Tabebuia spp, Pau d'arco, Tanacetum balsamita L., Costmary, Tanacetum
parthenium, Feverfew, Tanacetum vulgare, Tansy, Taraxacum officinale, Dandelion Root, Terminalia arjuna,
Arjuna, Theobroma cacao L., Cacao, Thuja occidentalis, Thuja oil, Thymus vulgaris, Thyme, Tilia spp,
Linden, Trachyspermum ammi, Ajwain Seed, Tribulus terrestris, , Tribulus terrestris L, Trifolium pratense,
Red Clover, Trigonella foenum-graecum, Fenugreek, Trillium erectum, Beth Root, Turnera diffusa,
Damiana, Tussilago farfara, Colt's Foot, Ulmus fulva, Elm, Slippery, Uncaria tomentosa, Cat's Claw, Urtica
dioica, Stinging Nettle, Vaccinium macrocarpon, Cranberry, Vaccinium myrtillus, Bilberry, Vaccinium spp,
Blueberries, Valeriana officinalis, Valerian root, Vanilla planifolia, Vanilla, Verbascum spp., Mullein,
Verbena hastata, V. officinalis, Vervain, Veronica officinalis, Speedwell, Vetiveria zizanoides, Vetiver Oil,
Viburnum opulus, Cramp Bark, Vinca minor, Periwinkle, Viola spp, Violet Leaf, Viscum coloratum, V.
album, Mistletoe, Vitex agnus-castus, Chaste Tree, Vitis vinifera, Grapes, Withania somnifera,
Ashwagandha root, Yucca spp, Yucca Root, Zanthoxylum spp, Prickly Ash, Zea mays, Corn Silk, Zingiber
officinale, Ginger Root

The chemical components being isolated or separated away from the plant material using this purification methodology includes phytocompounds, alkaloids, proteins, estragoles, anthocyanins, anthraquinones, bitters, cardiac glycosides, coumarins, cyanogenic glycosides, flavonoids, glucosilinates, minerals, mucilage, phenols, saponins, tannins, terpenes, vitamins, volatile oils, and the like. The methodology of the present invention provides for a highly purified product having greater than about 55% (e.g., 55%, 60%, 65%, 70%, 75%, 80%, 85%, 90%, 95%, 100%) by weight of the chemical component. Such chemical components can be any component derived from the starting plant material described herein and utilizing the methodology described herein.

The methodology can include an optional pretreatment step. The pretreatment step can be done with an acid to remove fatty acids or with a solvent to remove non-polar components such as chlorophyll, and structural and non-structural carbohydrates. Examples of fatty acids include chlorophyll, saponins, flavonoids, tannins, terpenes, iridoids, monoterpenoids, triterpenoids, phenolic compounds, glucosinates. These can be summarized as secondary metabolites. Such carbohydrates include, for example, cellulose, oligosaccarhides, polysaccharides, glucose, fructose, mannose, galactose, xylose, arabinose, xylan, glucuronoxylan, arabinoxylan, glucomannan, xyloglucan, mannan, glucans, pectin, plant gums, fructans, galactose, ribose, maltose, lactose, raffinose, fructooligosaccharide, manaoligosaccharide, inulin, and starch.

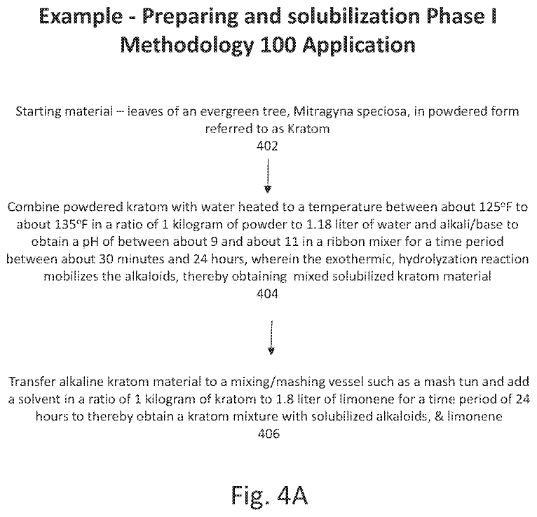

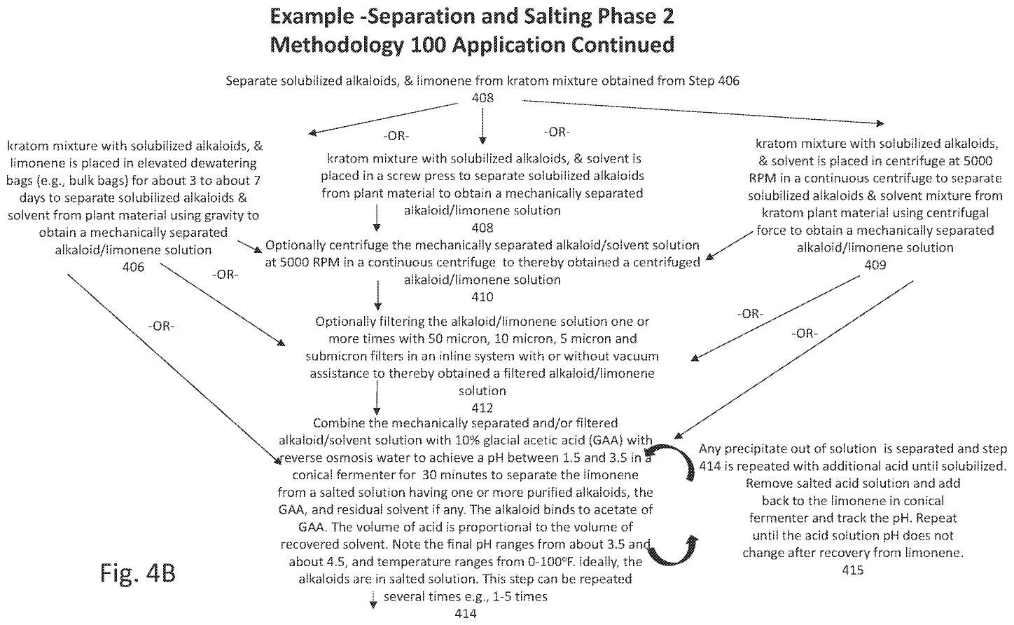

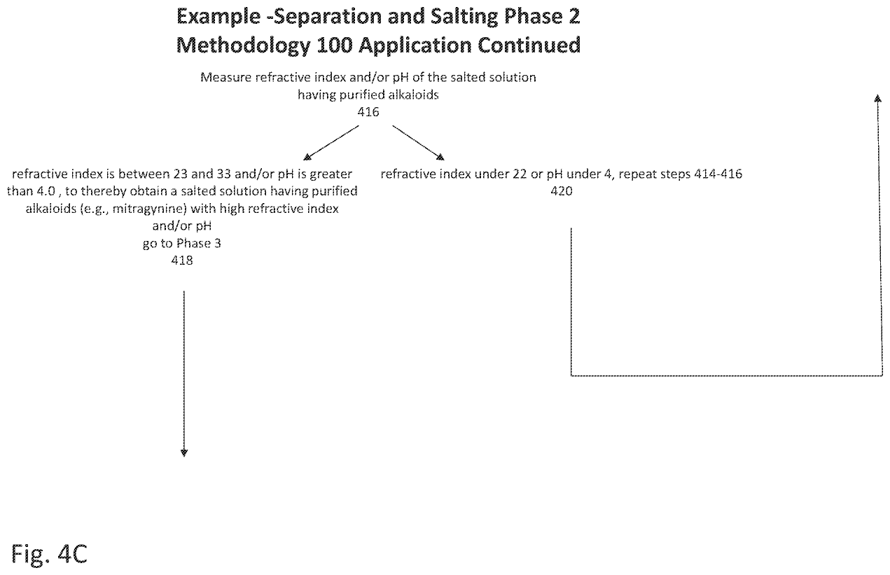

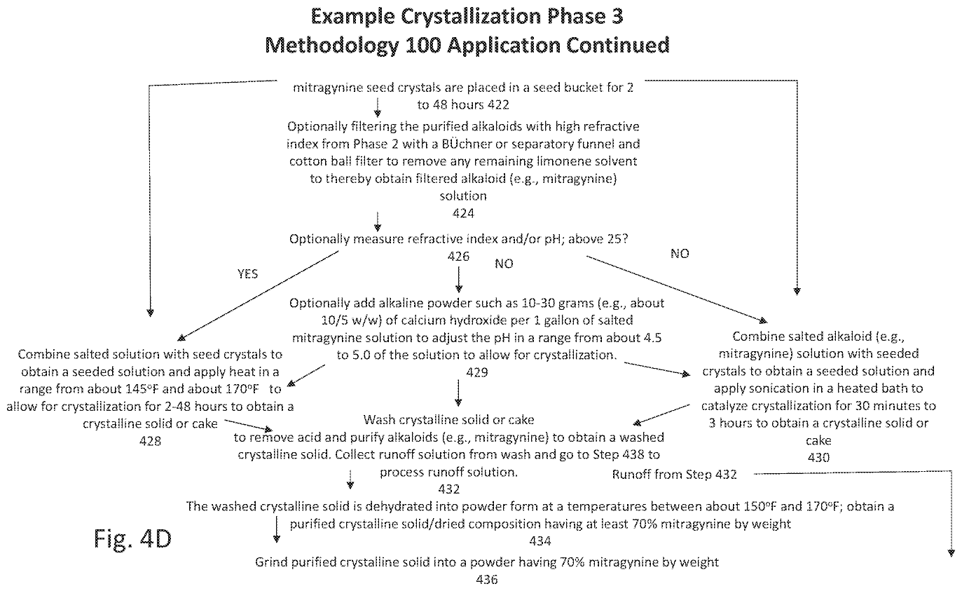

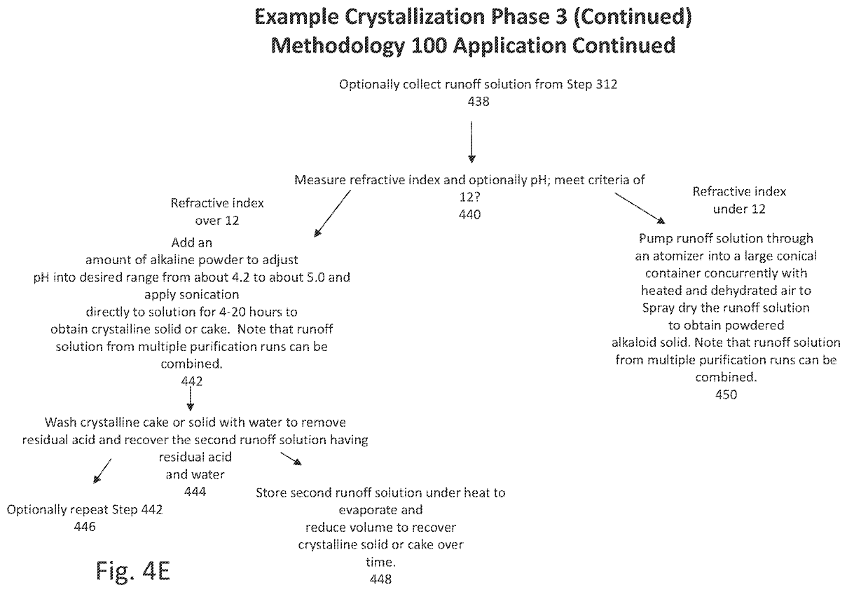

Methodology 100 shown in FIG. 1 and FIG. 4A includes combining the plant starting material with water and a solubilizer (e.g., a base or acid) to begin the solubilization of the alkaloids. Step 104 or Step 404. This step, Step 104, involves conditions to provide a exothermic hydrolyzation reaction to release chemical components (e.g., alkaloids) from the plant material. This step can be done in a mixer or similar device. Examples of such mixers include a ribbon mixer (Munson Ribbon blender New York New York USA), commercial mixers, cement mixer, mortar mixer, commercial blenders, and the like. In a particular embodiment, shown in FIG. 4A, powdered kratom or Mitragyna speciosa is combined with water heated to a temperature between about 100° F. to about 200° F. (e.g. 125° F. to about 135° F.), in a ratio of 1 kilogram of powder to 1.18 liter of water and base to obtain a pH of between about 9 and about 11 in a ribbon mixer for a time period between about 30 minutes and 24 hours, wherein the exothermic hydrolyzation reaction to release chemical components (e.g., alkaloids), thereby obtaining mixed alkaline kratom material having e.g., 4-70% moisture (e.g., 5% 10%, 20%, 30%, 40%, 50%, 60%, 70%). In an embodiment, 320 kilograms of plant material (e.g., kratom) is combined with an amount of water ranging between about 90 and 120 gallons (e.g., about 90, 95, 100, 105 110, 115, 120 gallons) or about 340 liters to about 454 liters of water heated at about 125-135° F. Heating assists in catalyzing the reaction to release the chemical components from the plant material in less time.

Temperature and solubility (e.g., alkalinity when using a base) allow for the hydrolyzation exothermal reaction to release the alkaloids. Temperature can be applied to the water before it is added to or to the mixture after the water is added. Heating the mixture allows for the alkaloids to be released at an increase rate and is optional. The temperature ranges from about 100° F. to about 200° F. (e.g. 125° F. to about 135° F.). Heat can be applied using a heater. Examples of such devices include heat exchangers, including falling film heat exchangers and tubular heat exchangers. Heat exchangers are able to deliver heat as well as cool the plant material. In an embodiment in which a heat exchanger is not used, a heater/oven or other device can be used.

The methodology of the present invention includes solubilization steps and salting steps. In an embodiment, a solubilizer is a base that is used to solubilize the one or more chemical components in the starting plant material in a non-polar solvent (see Step 104, and Step 404) and an acid is used to salt or polarize the chemical components (see Step 214 and Step 412). In another embodiment, an acid can be used for solubilization and a base can be used for the salting step.

Solubility can occur with the addition of a solubilizer, such as any acid or base, including “green” or food grade acid or base. As used herein, “green” acids or bases are those deemed safe by the USDA or similar organization, depending on the country. As used herein, “solubilized compounds” refers to the chemical components being dissolved in liquid or put into solution. In an embodiment, acid or base (e.g., solubilizer) is added in a sufficient amount and concentration to allow the chemical component to dissolve or solubilize without denaturing it. Any acid or base can be used to adjust the pH to ranges described herein to solubilize the component. Examples of such bases include calcium hydroxide, sodium carbonate, sodium hydroxide, potassium hydroxide, potassium carbonate or a combination thereof. In an embodiment, the base is a food grade, mineral base. In an embodiment, Step 404, calcium hydroxide is used and reacts with carbohydrates in plants and allows the alkaloids to be mobile/disassociate. Similarly, examples of acids that can be used for the present invention include glacial acetic acid, hydrochloric acid, sulfuric acid, nitric acid, carbonic acid, hydrofluoric acid, citric acid, phosphoric acid, ascorbic acid or a combination thereof. Other acids or bases, previously known or later developed, can be used in the steps of the present invention so long as they solubilize the chemical component under conditions described herein. The present invention can also utilize a Lewis acid or Lewis base. A Lewis acid is a chemical species that contains an empty orbital which is capable of accepting an electron pair from a Lewis base to form a Lewis adduct. A Lewis base, then, is any species that has a filled orbital containing an electron pair which is not involved in bonding but may form a covalent bond with a Lewis acid to form a Lewis adduct. The volume and concentration of the acid or base used to solubilize the chemical component at the desired pH will depend on the starting pH of the solution, the operating temperature of the equipment, and/or the volume of the solution being brought to the proper pH. The concentration of acid or base will depend on the particular acid or base being used and the composition (e.g., liquid or powder forms) but ranges between e.g., about 0.002M to about 0.00002M. The choice of the acid or base and its concentration should be one that does not denature the chemical component in the mixture. In an embodiment, to solubilize the component, the base adjusts the pH of the mixture to obtain a resulting pH in the range equal to or between about 9.0 and about 11 (e.g., about 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, or 11)). When using acid to solubilize the chemical component in the plant material, the acid adjusts the pH of the mixture to obtain a resulting pH in the range of equal to or between about 1.0 and about 5.0 (e.g., about 1.0, 1.5, 2.0, 2.5, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or 5.0). In an embodiment, solubilization of the mixture refers to the chemical component or alkaloid being mostly solubilized or in solution. In another embodiment, solubilization refers to the solution having least about 75% (e.g., 75%, 80%, 85%, 90%, 95%, 96%, 97%, 98%, 99%, or 100%) of the chemical component solubilized. Once the chemical component is solubilized, it is referred to as a “solubilized chemical component” or “solubilized alkaloid” or in the instance of using kratom as the starting material a “solubilized kratom material” or “solubilized mitragynine.” Once the solubilizer is added to the water and plant material and has the proper moisture content, one can proceed to the next step, Step 106 or Step 406. Adding a solubilizer to the starting material results in a “mixed solubilized plant material” or a “plant mixture.”

In an embodiment, a base is used with kratom as the starting material. See Steps 402 and 404. In this case, it takes a hydrogen ion off the amine functional group of the mitragynine which allows the mitragynine to become a similar polarity as limonene (e.g., racemic mixture that includes d-limonene and/or 1-limonene). Using the principle of like dissolves like, this allows the mitragynine to effectively solubilize into the solvent, as described herein. Step 406.

In an embodiment, water is added such that the mixed solubilized plant material has a moisture content of between about 1% and about 70% (e.g., about 1%, 2%, 3%, 4%, 5%, 6%, 7%, 8%, 9%, 10%, 11%, 12%, 13%, 14%, 15%, 20%, 25%, 30%, 35%, 40%, 45%, 50%, 55%, 60%, 65%, 70%), and in an embodiment has a content of between about 4% and about 6%. The moisture content depends on the ratio of liquid (water and base/acid) to plant material. The moisture content can be measured using a moisture meter. Any moisture meter can be used so long it is suitable for measuring moisture in a plant material. Moisture meters are commercially available, from e.g., Aroya SOLUS Soil Moisture Meter from Forever Green Indoors (Seattle WA, USA).

Step 106 or Step 406 of methodology 100 involves combining the plant material and a solvent. When the solvent is added, the mobile alkaloids of the plant mixture are further solubilized. This can be done in a mixing and/or mashing vessel such as a mash tun. The mash tun allows the mixed solubilized (alkaline (or acidic)) plant material to be mixed with a solvent to produce a “mash” type mixture. Such a vessel with a screen used for this mixing can include screens for filtering larger plant material. A 275 gallon IBC tote from Uline is commercially available and can be used for this step. Addition of the solvent in Step 106 or Step 406 results in a plant mixture with solubilized chemical components (e.g., alkaloids) & solvent.

The solvent used in this step can be any solvent that allows for further solubilization of one or more chemical components from the plant material. In an aspect, the solvent is added in a time frame ranging from 1 minute to about 30 minutes after adding the solubilizer in Step 104 or Step 404. In an embodiment, the solvent (e.g., limonene) is added before the mixture cools or as soon as possible after the exothermic reaction in Step 104 or Step 404.

A solvent can be any non-polar chemical substance that dissolves one or more of the chemical components in the mixture. Solvents of the present invention include those with a dielectric constant below about 5. Examples of such non-polar solvents include green and non-green solvents. Solvents for the present invention can also be food grade solvents as well. As used herein, “green” solvents are those deemed safe by the USDA or similar organization, depending on the country. Examples of green solvents include Limonene, p-cymene, pinene, cyclopentyl ethyl ether, ethyl lactate, 2-methyloxolaine, glycerol, Isopopanol, n-propylacetate, i-propylacetate, 1-butanol, 2-butanol, sulpholane, anisole, ethylene, propylene carbonate, dimethyl carbonate, tert amyl methyl ether, methyl laurate, 2-methyltetrahydrofuran, 2,2,5,5-tetramethyloxolane, cetyltrimethylammonium chloride, dimethyldodecylamine oxide, sodium dodecyl sulfate, dimethoxyethane, methylene chloride, polyethylene glycol and combinations thereof. Other solvents including non-green solvents can be used in the methodology of the present invention and include e.g., ethanol, methanol, petroleum ether, n-hexane, chloroform, methyl chloride, ethyl acetate, hexane, toluene, naptha, xylene, and combinations thereof. Using green solvents is more labor intensive and less effective. However, using green solvents is better for the health of workers doing the purification, easier to dispose of and healthier for consumers ingesting it. Despite using green solvents, the present invention still results in a highly purified product. Similar solvents now known or later discovered or developed can be used with the present invention. In an embodiment, green solvents or food grade solvents used in the present invention meet Hanson solubility parameters (Green Solvents, Hansen Solubility Parameters, https://www.hansen-solubility.com/HSPiP/green-solvents.php) or Gauss polarity parameters https://en.wikipedia.org/wiki/Polarization_density#Guass's_law_for_the_field_of_P. Solvents later discovered or known can be also used in the methodology of the present invention.

In an embodiment, kratom material having 4-6% moisture content is transferred to a mash tun and a solvent in a ratio of 1 kilogram of kratom to 1.8 liter of d-limonene is added and the mash processes occurs for a time period of 24 hours, repeated twice more for 2 hours each, to thereby obtain a kratom mixture with solubilized alkaloids and solvent. See Step 406 of FIG. 4A. In yet another embodiment, approximately 75-85 gallons (about 283 liters to about 321 liters) of limonene is added to about 160 kilogram of kratom material. In this embodiment, limonene is slightly non-polar and solubilizes the alkaloid/chemical component by forming an ionic bond.

### Phase 2: Separation and Salting

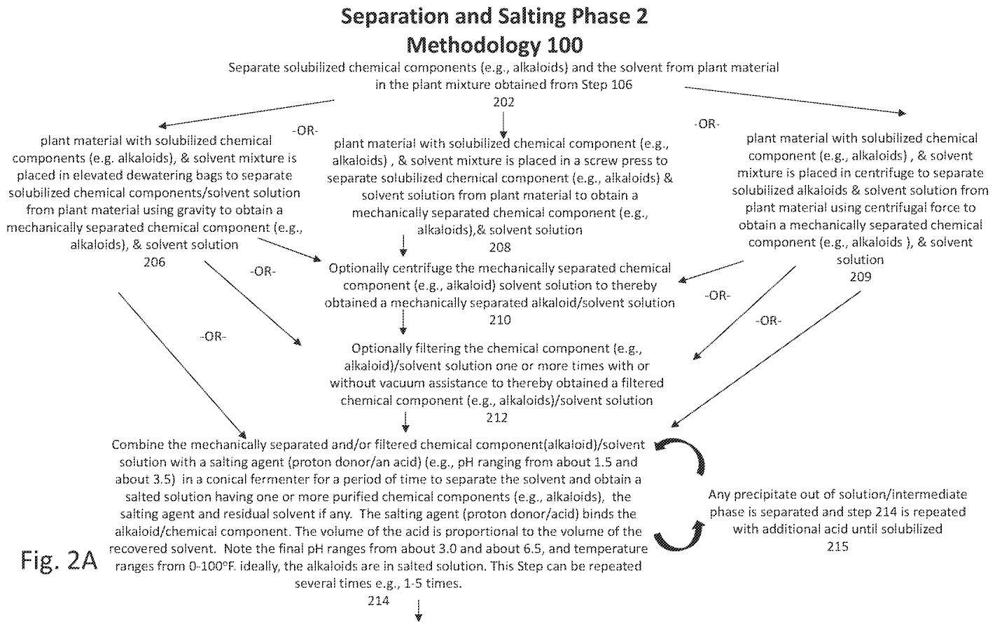

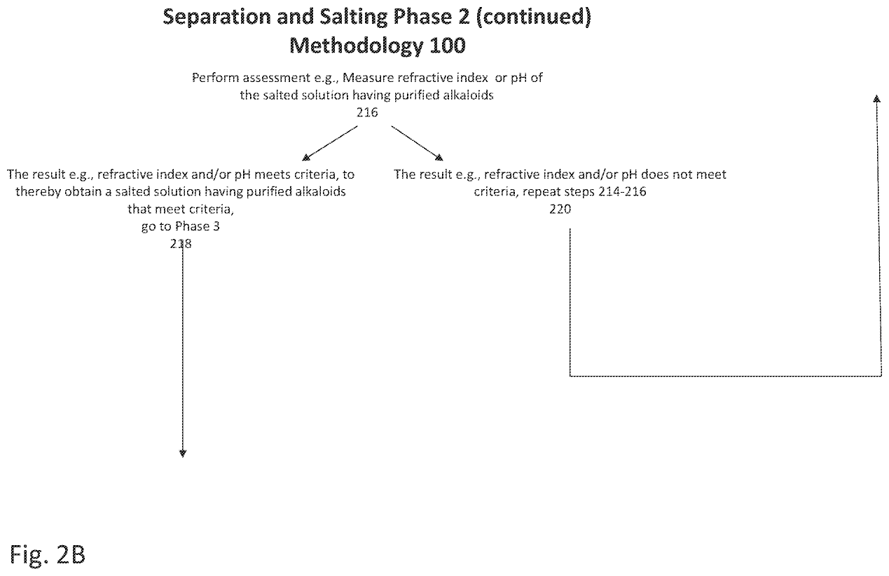

Phase 2 involves two basic steps: mechanical separation of the plant material (see Steps 202-212) and salting the solubilized chemical components (see Steps 214-215). The first basic step of Phase 2 involves mechanical separation (see Steps 202-212) of the plant material in the plant mixture from Step 106 having plant material, solubilized chemical components/alkaloids, and solvent, as shown in FIGS. 2A and 2B, and also in FIGS. 4B and 4C.

Mechanical separation in the steps of the inventive methodology can occur in any number of ways using commercially available equipment. Mechanical separation techniques include, for example, gravity, pressing, filtration, sedimentation, centrifugation, or strainer. Mechanical separation equipment includes filters, screens, screw press, drip bins), centrifuges, filter press, and the like (e.g., to capture the solution when gravity or force (e.g., centrifugal or vacuum) is applied). In an embodiment, the mechanical separation technique used in the present invention includes hoisting dewatering bags and allowing gravity to separate the solubilized chemical components (e.g. alkaloids) & solvent solution from the plant material. See Step 206 or Step 406. In this case, the solution collects into a container. In another embodiment, a screw press is used to mechanically separate the solution having chemical components (e.g., alkaloids), & solvent solution from the plant material. See Step 208 or Step 408. A screw press applies force to squeeze the solution out of the plant material against a screen or filter and the solution is collected through the screen for collection. Screw presses are commercially available from, for example, Vincent Corporation (Model No. CP-6, Tampa, Florida USA). If the gravity method is utilized, any surplus can be process through the screw press to maximize recovery. In yet another embodiment, centrifugation can be used to separate plant material from the chemical components (e.g., alkaloids) & solvent with centrifugal force. Any method of mechanical separation can be used so long as the plant material is separated from the chemical component (e.g., alkaloids) & solvent solution.

After the plant mixture is separated from solubilized chemical components (e.g. alkaloids) & solvent by the dewatering bags in Step 206 or 406, screw press in Step 208 or Step 408, or the centrifuge in Step 209 or 409, the resulting solution is mechanically separated chemical component (e.g. alkaloids) & solvent solution.

Mechanical separation methods can be repeated or combined if needed. For example, in an embodiment, once the plant mixture with solubilized alkaloids has been processed by the dewatering bags in Step 206 or 406, screw press in Step 208 or Step 408, or the centrifuge in Step 209 or 409, the mechanically separated alkaloid solution can further be optionally processed by centrifugation as shown in Step 210 or Step 410.

In embodiment, for steps 209 and/or 210, a continuous centrifuge is used at about 5000 RPM for 30 minutes to about 90 minutes and feeds from the screw press to further separate the solids from the liquids and provide a high concentration of the chemical component (e.g., alkaloids)/solvent solution. Centrifugation can be done with or without a built-in filter. In another embodiment, centrifugation in Methodology 100 can also use a decanter centrifuge @ 3000 RPMs (500-3000 RPMs) or 1000-3000 G force. Centrifuges are commercially available, and for example, can be obtained from Alpha Laval (Beverly, Massachusetts USA). Any centrifugation settings can be used so long as the centrifuge separates the plant material from the chemical components (e.g., alkaloids)/solvent solution.

If the mechanically separated solution having solubilized chemical components (e.g., alkaloids) & solvent still has some plant material then it can optionally be further filtered with or without vacuum assistance in Step 212 or Step 412. See FIG. 2A or 4B. The solution can be filtered one or more times with varying size filters. In an embodiment, the solution is filtered by passing through an array of filters having a size of 50 microns, 10 microns, 5 microns, absolute sub-micron. The filtration system aids in filtering out plant material and any remaining solids. In an embodiment, the mechanically separated solution from Steps 206, 208, 209 and/or 210 or Steps 406, 408, 409 and/or 410 is filtered at least three times using the following size filter in the following order—25 microns, 10 microns, and 1 micron. In other embodiments, centrifuges can also have a filter and so this step can be combined with the centrifuge steps, described herein.

Filtration can be vacuum assisted. In such an embodiment, a negative pressure vacuum can be used to pull the fluid through the filter. Alternatively, positive pressure directed air can be used to push the fluid through one or more filters.

The separation steps result in a separation of soluble chemical components (e.g., alkaloids) in a solvent from the plant material. The plant material is discarded. The remaining solution has the solvent, and the solubilized chemical components (e.g., alkaloids). The methodology then proceeds to the salting/precipitation Step 214 or Step 414.

The separated (e.g., mechanically separated) and/or filtered solution is transferred, in Step 214 or Step 414, to a vessel in which the solution is salted or precipitated. In the salting/precipitation step, a salting agent (e.g., acid or proton donor) is added to bind and/or precipitate the purified chemical components (e.g., alkaloid) and separate all or most of the solvent from the purified chemical component (e.g., alkaloid) solution. The addition of the salting agent (e.g., acid or proton donor) results in a salted solution having one or more purified chemical components (e.g., alkaloids), the salting agent and residual solvent, if any.

Various vessels used for the salting/precipitation Step 214 or Step 414 so long as the salting agent can be added and/or the separation of the solvent can occur. Such vessels include those used for phase separation. Examples of such include a shear mixer, an agitator, a static mixer, a percolator, conical fermenter and the like. In an embodiment, a vessel is a conical fermenter, which is commercially available from Hengcheng or Stout, Model No. 7BBLCV (Shandong China).

In an embodiment, salting/precipitating occurs by adjusting the pH of the solubilized solution into the isoelectric range of the plant involved. See Step 214 or Step 414. The isoelectric range for plants, in general, is a pH between about 3.0 and about 6.5 (e.g., about 3.0, 3.5, 4.0, 4.5, 5.0, 5.5, 5.6, 5.7, 5.8, 5.9, 6.0, 6.1, 6.2, 6.3, 6.4, or 6.5). The isoelectric range can depend, for instance, on conditions such as salt, the starting material, the type of chemical component (e.g., alkaloid), the charge of the component, the amino acids that make up the component, and the ionic strength of the solution to which the chemical component (e.g., alkaloid) has been subjected. Adjusting the pH to the aforementioned isoelectric range can be performed by adding either an acidic solution or a basic solution. If base was added in Steps 104 or 404 to solubilize the chemical component, then acid can be added in Steps 214 or 414 to salt the chemical component. Similarly, if an acid was added to solubilize the chemical component, then base can be added to salt the chemical component. Any acid or base (e.g., a polarity changer) can be used to adjust the pH to ranges described herein to precipitate the chemical component. As described above, examples of such bases include calcium hydroxide, sodium carbonate, sodium hydroxide, potassium hydroxide, potassium carbonate, calcium carbonate or a combination thereof. Similarly, examples of acids that can be used for the present invention include glacial acetic acid (GAA), hydrochloric acid, carbonic acid, sulfuric acid, nitric acid, sulfuric acid, hydrofluoric acid, citric acid, phosphoric acid, ascorbic acid or a combination thereof. Other acids or bases, including Lewis acids and bases, as described herein and those previously known or later developed, can be used in the steps of the present invention so long as they precipitate or bind with the chemical component (e.g., alkaloid) as described herein. The final range of the salted solution has a pH between about 3.0 and about 6.5 (e.g., about 3.0, 3.5, 4.0, 4.5, 5.0, 5.5, 5.6, 5.7, 5.8, 5.9, 6.0, 6.1, 6.2, 6.3, 6.4, or 6.5

In an embodiment, when there is a greater difference in pH between the starting pH of the salting agent (e.g. GAA) and ending pH, the more alkaloid is in the solution that is retrieved through phase separation at the bottom of the conical fermenter. In practice, in Steps 414, the acid is poured through at the top of the conical fermenter having solubilized chemical components (e.g., alkaloids)/solvent solution. As the acid moves through, the acid forms a covalent bond with the alkaloid and forms the salted solution which goes to the bottom of the conical fermenter. The solvent stays at the top. There can be an intermediate phase with both alkaloid and solvent. In one embodiment of Step 414, GAA acid is added at pH of about 2.25 with the final salted solution having pH of about 4-4.5.

In step 215 or 415, the intermediate phase having the emulsion or mix of solvent and alkaloid can be reprocessed in a smaller conical fermenter with additional salting agent (e.g., GAA).

During step 214 or 424, in an embodiment, the volume of the acid is proportional to the volume of the recovered solvent.

A spray ball device can be used to emit the salting agent/polarity changer—the acid or base. The volume and concentration of the acid or base used will depend on the starting pH of the solution, and the volume of the solution being brought to the proper pH. In another embodiment, the salted solution has least about 75% (e.g., 80%, 85%, 90%, 95%, 96%, 97%, 98%, 99%, or 100%) of the one or more chemical components solubilized. In an embodiment, 100 ML of GAA 10% solution is added per every one gallon of alkaloid solution. Pure GAA can be purchased and diluted in reverse osmosis water. During the salting step, protonation occurs and the polarity changer binds to the alkaloids and/or chemical components in the salted solution.

The time period for the salting step can be from 10 minutes to 120 minutes, and preferably for about 60 minutes. During this time, the mixture undergoes phase separation. Due to the composition or density of the phases (e.g., salting agent/chemical components and solvent), the phases separate. The higher density liquids will move to the bottom of the conical fermenter and the comparatively less dense liquids will rise to the top. In an embodiment, the solvent used in Phase 1 floats to the top and the salting agent/alkaloid solution (“salted solution”) goes to the bottom. See Step 214 The density of Limonene is about 0.8411 g/cm³ and the density of GAA is 1.05 g/cm³ and is denser with the alkaloids bound thereto. For example, the density of mitragynine is 1.2 g/cm³. In a specific embodiment, Limonene floats to the top and the GAA/alkaloid solution (“salted solution”) goes to the bottom. See Step 414.

In an embodiment, the choice of the salting agent or polarity changer also includes its density and that the salting agent or polarity changer's density is greater than that of the solvent chosen for the methodology 100.

Assessment or analysis of the salted solution is performed to determine if it meets a set criteria associated to the purity of the solution. Any number of analyses can be carried out to determine if the solution has the desired purity of the chemical component. In an example, the refractive index of the sample of the salted solution, or in an embodiment the alkaloid/glacial acid solution, is measured. See Step 216 and Step 416. The refractive index is a measure of the bending of a ray of light when passing from one medium into another. A more technical definition of the Index of Refraction is a value calculated from the ratio of the speed of light in a vacuum to that in a second medium of greater density. Basically, the refractive index determines how much the path of light is bent, or refracted, when entering a material. When the refractive index of the salted solution meets criteria set by the chemical component being purified, then one can proceed to Phase 3 of the methodology. When the assessment (e.g., refractive index) of the salted solution is below the established criteria, then the salting step in the conical fermenter can be repeated until the criteria is met. In an embodiment, the refractive index threshold is between about 23 and 33 (e.g., about 23, 24, 25, 26, 27, 28, 29, 30, 31, 32, or 33). When the refractive index is under about 22, then the salting step, Step 214 or Step 414, is repeated.

Refactor meters are commercially available, from, for example, Mettler Toledo (Columbus, Ohio, USA).

The refractive index is an indirect measurement of the level of purity of the chemical components/alkaloid in the salted solution. Purity levels can be accomplished in other ways. For example, the purity of the chemical components/alkaloid of salted solution can be measured directly or indirectly. Examples of other suitable analysis include High-performance liquid chromatography (HPLC), Ultra Performance Liquid Chromatography, spectrometry, gas chromatography-mass spectrometry, photodetectors, photosensors, lasers, light scattering detection, and the like. Any type of analytical equipment that could replace a refractive index meter in the inventive methodology.

Additionally, in an embodiment, pH measurements using a pH meter can be used to determine if the salted solution has a level of purity to go to Phase 3, crystallization stage. PH and/or other analyses can be used in place of or in conjunction with the refractive index measurement. In an embodiment, the pH criterion to move to Phase 3 is a pH ranging from about 3.0 to about 6.5 (e.g., about 3.0, 3.5, 4.0, 4.5, 5.0, 5.5, 6.0, 0.6.5) or greater. A pH of 3.0 or greater, then one can move on to Phase 3, Step 304. In an embodiment, two or more criteria are met e.g., the refractive index is between about 23 and about 33 and the pH is greater than about 4.0 to move on to Phase 3. In another embodiment, only one direct or indirect purity criterion needs to be met. In one aspect when the pH is less than about 3.0, steps 214-216 or 414-416 can be repeated. In an embodiment, refractive index and/or pH is used in Step 416 because it is easy and fast in an industrial and/or commercial setting.

At this point, the salted solution consists largely of the salting agent (e.g., polarity changer) and the chemical component (e.g., alkaloid) being purified. In an embodiment, the salted solution may have a small amount of the solvent (e.g., residual solvent) which can be separated/removed in the next phase, Phase 3.

### Phase 3: Crystallization

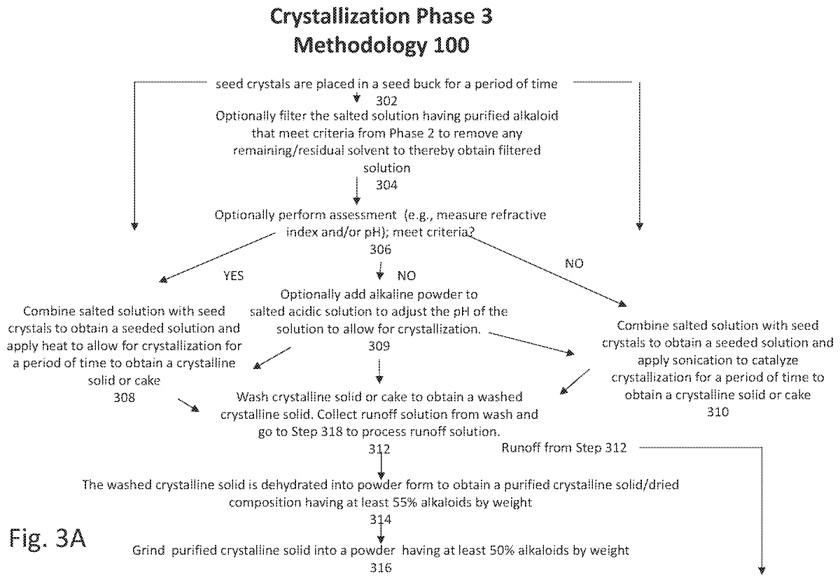

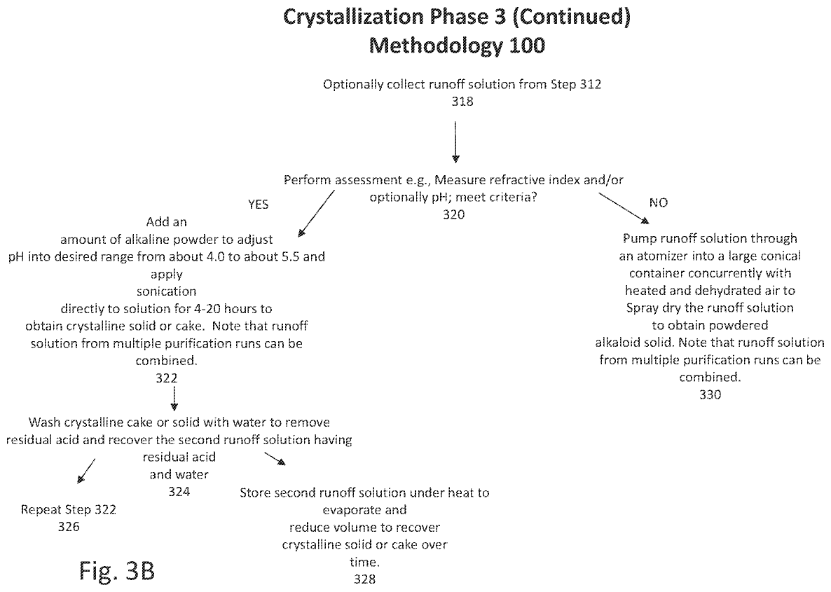

Referring to FIGS. 3A-B and FIGS. 4D-E, the salted solution that meets the desired criteria (e.g., measured with a refractive index and/or pH) undergoes the crystallization phase. A crystal is a solid material with atoms and molecules that are arranged in a solid ordered substance and a consistent, organized and repeating pattern, creating geometrical shapes. Additionally, a crystal is a homogeneous solid substance having a natural geometrically regular form with symmetrically arranged plane faces. In an embodiment, crystals can be formed from a solution and through evaporation or when liquids cool.

A seed crystal can be used to facilitate crystal formation. A seed crystal refers to a small piece of single crystal or polycrystal material which forms the base on which the rest of the crystal lattice/structure forms. Since the crystal structure is a repeated, geometrically regular shape, the seed crystal promotes the formation of additional repeated lattice structures. Intermolecular forces between the separate molecules and form a basis for a crystal lattice. Often, this phase transition from solute in a solution to a crystal lattice will be referred to as nucleation. Nucleation refers to the first step in a thermodynamic phase change that results in an organized and/or repeating assembly and/or organization of molecules that make up a crystal. It is governed by concentration, pH, time, pressure, and temperature, but is a random process, in such that two identical systems can have different nucleation rates. Crystallization includes two stages: nucleation and crystal growth. Accordingly, the term “crystallization” as used herein refers to any stage in the process including both “nucleation” and “crystal growth.” The term “crystals” includes elements in any stage of the crystallization process. The seed crystal is typically the same or similar to the crystal structure that is being grown. The seed crystals sit for a time period, for example, from about 2 hours to about 48 hours (e.g., about 2, 3, 4, 5, 6, 7, 8, 9, 10, 12, 15, 20, 24, 25, 30, 35, 36, 40, 45, 48 hours). Step 302 of FIG. 3 or Step 422 of FIG. 4. In an embodiment, the vessel in which the seeding occurs is a suitable container or seed bucket. The use of a seed bucket optional and crystal seeds, either dried or moistened, can be placed just prior to the heating Step 308/428, alkaline power Step 309/429 or sonication Step 310/430.

In an embodiment, standardized mitragynine crystal or a crystal from a previous batch is used to seed when using kratom as the starting plant material. See Step 422.

In an embodiment, crystallization is a function, in part, of pH, temperature and pressure. In an embodiment, to promote crystallization, the starting pH is in a range from about 3.0 to about 7.0, a change/delta in temperature occurs e.g., increase temperature with a heater or decrease temperature with a chiller/cryogenics and/or low pressure is induced. A colder temperature form smaller crystals with higher purity and warmer temperature forms larger crystals with lower purity.

Phase 3 begins with removing any remaining or residual solvent, if any, from the solution having the alkaloid/chemical component and salting agent (e.g., polarity changer). Any filtering or separation process can be used to remove the remaining solvent. In an embodiment, a Buckner funnel and filter arrangement can be used to separate the residual solvent from the salted solution (having the alkaloid/chemical component being purified and the salting agent (e.g., glacial acid)). See Step 304 or 424. In a specific embodiment, any remaining solvent can be captured with a simple “cotton ball” filter to which the solvent adheres. See Step 304. This step is optional. In a particular embodiment, any remaining limonene solvent is separated with this filter to which the solvent adheres. Step 424, FIG. 4D. Other filters such as an impregnated filter can be used to remove the residual solvent. The filtering stage is optional. Other methods of removing the residual solvent can be used e.g., phase separation, centrifugation, spinning band distillation.

Once the solvent is removed (e.g., limonene), another assessment (e.g., the refractive and/or pH) measurement of the solution can optionally be taken again. Methods for determining the refractive index are described herein. In general, removal of the solvent increases the refractive index. When the assessment of choice meets the criteria, the solution is combined with the crystal seeds and heated. When the assessment of choice (e.g., refractive index or pH) does not meet the second set of criteria, then the filter solution is combined with crystal seeds and undergoes sonication. In an embodiment in Phase 3, the refractive index threshold is between about 23 and 35 (e.g., about 23, 24, 25, 26, 27, 28, 29, 30, 31, 32, 33, 34, or 35) and the pH is in a range from about 3.0 to about 7.0. When the threshold is met, then the next step in the methodology is Step 308 or Step 428, which is to heat the seeded solution. When the assessment of choice does not meet the criteria e.g., the refractive index is under about 25, then the sonication step, Step 310 or Step 420, is performed.

Accordingly, Phase 3 includes combining salted solution that meets the assessment criteria with seeding crystals from Step 302 or Step 422 to obtain a seeded solution. The crystal seeds are suitable for crystallization of the desired alkaloid. Crystallization is simply the process by which a crystal forms, e.g., where the atoms or molecules are highly organized into a lattice structure known as a crystal. Seed crystals from a previous batch can be used for future batches.

Various methods can be employed to promote crystallization. Examples include heat, pH adjustment, sonication and the like. Any method of crystallization and promotion thereof known in the art can be used.

In an embodiment, the seeded solution having that meets the assessment threshold or criteria is heated for a period of time to obtain a crystal solid or cake. The heated temperature ranges from 60° F. to about 200° F. (e.g., 160° F. to about 200° F.). Heat can be applied using a heater, heat exchanger or heat oven. Examples of such devices are described herein. The seeded solution sits for a time period, for example, from about 2 hours to about 48 hours (e.g., about 2, 3, 4, 5, 6, 7, 8, 9, 10, 12, 15, 20, 24, 25, 30, 35, 36, 40, 45, 48 hours). See FIG. 3A, Step 308 or FIG. 4, Step 428. In one aspect, the seeded solution is not being dehydrated, but heated. To prevent dehydration, the seeded solution can be covered.

In an embodiment, the seeded solution having an assessment that does not meet the threshold or criteria undergoes sonication with an ultrasonic liquid processors, also referred to herein as a “sonicator.” See Step 310 of FIG. 3A or Step 430 of FIG. 4D. A sonicator is device that emits ultrasonic energy to agitate particles in liquids and creates nanoparticles within the solution. In an instance, sonication catalyzes or promotes crystallization by orienting molecules in a homogeneous direction making crystal formation more likely. Sonicators can emit ultrasonic frequencies greater than 20 kHz. Sonicators are commercially available from, for example, Vevor ultrasonic TH-100A; 600 watts; 40 kilohertz (HK SISHUN TRADE CO., LIMITED, Hong Kong), or Qsonica sonicators (Newtown CT USA). Sonication occurs for a period of time sufficient to obtain a crystal solid or cake. The frequency at which the sonication occurs is greater than 20 kHz and in an embodiment is about 36 KHz and about 40 KHz. Sonication can be performed for a time period sufficient to allow for crystallization. In an embodiment the sonication time period ranges from 30 minutes to about 3 hours (e.g., about 30 minutes, 1 hours, 1.5 hours, 2 hours, 2.5 hours and 3 hours). This can be done optionally in a heated bath having a temperature that ranges from about 60° F. to about 200° F. (e.g., 160° F. to about 200° F.). Crystallization forms a crystalline solid or cake.

When using the sonication process, secondary alkaloids, isomers and chemical components can be obtained and may be desirable. The sonication process can produce nano-particles or smaller portions of the alkaloids. Examples of secondary alkaloids are included in Table 2. In one aspect, the sonicated crystal solution can be washed in order to obtain a crystalline solid or cake.

TABLE 2
Secondary alkaloids produced with present invention

ajmalicine, akuammigine, ciliaphylline, corynantheidine, corynoxeine, corynoxine A,
corynoxine B, epicatechin, 9-hydroxycorynantheidine, 7-hydroxymitragynine,
isomitraphylline, isomitrafoline, isopteropodine, isorhynchophylline, isospeciofoline,
mitraciliatine, mitragynine, mitragynine n-oxide, mitragynine oxindole B, mitragynine
pseudoindoxyl, mitrafoline, Mitraphylline, mitraversine, paynantheine, thrnchophylline,
speciociliantine, speciociliatine n-oxide, speciofoline, speciogynine, speciophylline,
stipulation, tetrahydroalstonine and their stereoisomers for the above

In yet another embodiment, crystallization can be promoted by adding an alkali to the salted solution that meets the aforementioned criteria to seed crystals for a period of time. See Step 309 of FIG. 3A and Step 429, FIG. 4D. The pH of the salted solution is increased to a pH that ranges from about 4.0 to about 7.0 (e.g., about 4.0, 5.0, 6.0, 7.0). Any base described herein for other steps can be used in this step as well. Once the pH is in range, then the basic, seeded solution from step 309/429 can be heated in Step 308 or 428 and/or undergo sonication in Step 310 or 430. Alternatively, no heat or sonication is added and the seeded solution treated with alkaline powder will eventually crystalize but will take more time as compared to that if heat and/or sonication was applied.

The crystalline solid or cake from Step 308/428 or 310/430 or 309/429 is washed using standard washing procedures and reverse osmosis water. See Step 312 or 432. The washing step can be repeated several times (e.g., 2, 3, 4, 5, 6, 7, 8, 9, or 10 times). Vacuum pressure can be applied to improve the speed of the wash. Vacuums are known in the art and commercially available. The washing process removes the salting agent (e.g., polarity changer or glacial acetic acid). In an embodiment, a filter can be used during the washing process wherein the filter captures the crystals and allows the water and the polarity changer and other contaminants to pass. In an embodiment, reverse osmosis water is used to wash the crystalline solid/cake and allows the filter captures the crystals and allows the glacial acid and other contaminants to pass through. The washed crystalline solid is a highly concentrated or purified product having greater than about 55% (e.g., 55%, 60%, 65%, 70%, 75%, 80%, 85%, 90%, 95%, 100) by weight of the chemical component (e.g., alkaloid).

In an embodiment, washing in Step 312/324 or 432/444 can be done with water. In other embodiments washing can also be performed with hydrogen peroxide, ethanol, alkaline alcohol solutions, acidic alcohol solutions, alkaline solutions, acid solutions and the like. Such washing step can be done by pouring the wash over the crystalline solid/cake or can be done in a device such as a reactor vessel, agitator, centrifuge, or with negative/positive pressure filtration.

Optionally, the runoff solution from step 312 or 432 can also be processed. Runoff solution in Step 318 or 438, FIG. 3B or 4E, can be collected from Step 312 or 432, FIG. 3A or 4D. The runoff solution includes e.g., excess salting agent (e.g., polarity changer) and wash fluid. The liquid is batch stored in a vessel. An assessment or analysis is performed to determine if the desired criteria are met. In an embodiment, the refractive index and/or pH of the liquid is measured. Step 320 or 440. Using the refractive index or pH, a base is added to the liquid to increase the pH to a range of about 3.0 and about 7.0. After the base is added, sonication, as done in Step 310 or 430 is performed for a time period in a range from about 4 hours and about 24 hours to thereby obtain a crystalline solid or cake. Step 322 or 442. This step can be repeated multiple times e.g., 1-5 times. See Step 326 or 446. After sonication, crystalline solid or cake is washed to remove residual acid. Step 324 or 444. Washing for this step can be performed as described herein for Step 312 or 432. Step 322 or 442 can be repeated if necessary. The second runoff solution from Step 324 or 444 is subjected to heat to evaporate the liquid to recover crystalline solid/cake for a time period ranging from about one to four weeks. Step 328 or 448. The runoff collected can be from one batch or multiple batches. The collected runoff that does not meet the refractive index criteria and/or pH criteria can be spray dried instead of crystallized. See Step 330. This runoff, in an embodiment, can be pumped to a spray dryer. The spray dryer includes an atomizer with pressurized gas, the liquid feed and heated gas. The liquid feed hits the pressurized gas to atomize the liquid which is then dried by the heated gas. This can be done by a commercial spray dryer or one that is commercially available (Anhydro CF-100 SPX Denmark). Example flow rates of the pressurized gas ranges from about 90 and about 125 psi, and the liquid feed of the runoff ranges from about 50 kg/24 hours to about 70 kg/24 hours. The heated temperature ranges from about 130° C. to about 190° C. The spray dried runoff results in an amorphous power having an amount of the chemical component ranging from about 3% to about 30% w/w.

Referring back to FIGS. 3A and 4D and Step 314 or 434, respectively, the washed crystalline solid can be placed in a dehydrator to remove moisture. See Step 314 or 434. A commercial dehydrator that removes moisture and/or adds heat. As such, several commercial dehydrators often have a dual function. Dehydrators work by circulating dry, heated air for a period of time. In an embodiment, the washed crystals are subjected to dehydration for a time period ranging between about 12 hours and 18 hours (e.g., about 12, 13, 14, 15, 16, 17, or 18 hours) and at a temperature between about 150° F. and about 170° F. (e.g., about 150° F., 155° F., 160° F., 165° F., or 170° F.). The dehydration process results in a dried crystalline composition.

The purified crystalline solid can be ground into a fine powder using grinding equipment known in the art. See Step 316 and 436. In an embodiment the purified crystalline solid/dried composition is ground into a powder using a hammermill, grinders, and the like. In an embodiment, the purified crystalline solid from Step 314 or 434 can be put into a solvent and spray dried if a highly purified amorphous solid is desired.

As described herein, the methodology of the present invention provides for a highly purified product having greater than about 55% (e.g., 55%, 60%, 65%, 70%, 75%, 80%, 85%, 90%, 91%, 92%, 93%, 94%, 95%, 96%, 97%, 98%, 99%, 100%) by weight of the chemical component. The concentration of the chemical component can be determined in one instance by High Performance Liquid Chromatography (HPLC). Any process known in the art can be used to determine the identity and amount of a chemical component. High Performance Liquid Chromatography (HPLC) is a process of separating components in a liquid mixture. A liquid sample is injected into a stream of solvent (mobile phase) flowing through a column packed with a separation medium (stationary phase). Sample components separate from one another by a process of differential migration as they flow through the column. As bands emerge from the column, flow carries them to one or more detectors which deliver a voltage response as a function of time. This is called a chromatogram. For each peak, the time at which it emerges identifies the sample constituent with respect to a standard. The peak's area represents the quantity. Using this technology, methodology 100 results in a high concentration by weight of about 55% or greater.

As can be seen in Example 4 from the independent third-party results, the amount of alkaloids in the purified crystalline solid product is greater than about 90%. Accordingly, the chemical component, as demonstrated by test results, are well within the range from about 55% to about 100%. The methods of the present invention result in an efficient highly purified product despite the commercial and industrial processing of large quantities.

Accordingly, the ratio of starting plant material to purified crystalline product ranges between about 100:1 to about 150:1 with a purification of a chemical component such as one or more alkaloids ranging from about 55% to about 100% (e.g., at about 55%, 60%, 65%, 70%, 75%, 80%, 85%, 90%, 95%) by weight. The analysis shows that crystallization using heat results in a highly pure alkaloid composition. In Example 4, Table 3, the total alkaloid in the final product is 96.2% wherein 91.88% is mitragynine and 4.33% are secondary alkaloids. Table 4 shows similar results with 94.5% total alkaloids with 89.23% mitragynine and 5.24% secondary alkaloids. Both sets of analysis show a highly pure product, well above 55%, performed in an industrial bulk processing facility. The analysis from Table 5 shows that crystallization using sonication also results in a highly pure alkaloid composition. In Table 5, the total alkaloid in the final product is 97.4%.

The amount of purified alkaloids obtained by heating-promoted or sonication-promoted crystallization results in about the same amount in total alkaloids by weight. However, it was determined, surprisingly, that crystallization by sonication results in an increase of secondary chemical components (e.g., alkaloids) and a decrease in the primary chemical component (e.g., primary alkaloid) (e.g., the alkaloid present in an amount greater than any other alkaloid in the composition). Secondary alkaloids have been traditionally difficult to purify in a commercial or industrial basis. As shown in Example 4, the sonicated purified crystalline solid product has 80.87% mitragynine and 16.54% are secondary alkaloids with over 11.41% Speciocilatine, 2.65% Speciogynine and 2.48 Paynantheine. This is a surprising result. The method of crystallization, heating vs sonication, resulted in a different composition of secondary alkaloids. Secondary alkaloids are desired in certain circumstances such as for treating various conditions. For example, Speciocilatine, Speciogynine and/or Paynantheine can used for medical purposes (e.g., pain management and/or to treat opioid addictions). All sets of analysis show a highly pure product, well above 55%, performed in an industrial bulk processing facility.

Accordingly, the present invention includes a dried composition or product having an amount of the primary chemical component (e.g., mitragynine) of at least about 70%, 75%, 80%, 85%, 90%, or 95% w/w and an amount of one or more secondary chemical components (non-mitragynine alkaloids) of at least about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 240%, 25%, 26%, 27%, 28%, 29% or 30% w/w.

### EXEMPLIFICATION

### Example 1: Phase 1: Preparation and Solubilization of Plant Material

### Methodology

1. Clean and sanitize mash tuns for production.—

2. Empty De-watering bags (super sacks) and rinse with water, also sanitize bags.

3. Clean and sanitize ribbon mixer.

4. Raw powdered kratom in an amount of 320 kilograms was combined with 100 gallons water heated to a temperature of 125° F. and 135° F. with 104 kilos base in a ribbon mixer (Munson Ribbon blender New York New York USA). The pH of the solution was 9.5. The mixer was run with dry ingredients for a minimum of 15 minutes and once water is added, the ribbon mixer mixed for an additional 10 minutes.

5. The hydrolyzation exothermic reaction released alkaloids.

6. The mash was checked with a moisture meter. Temperature was also checked. Temperature should be between 155-165.

7. Can be adjusted with water if the moisture meter is not between those perimeters.

8. Moisture meter should read between 4-6% on the dial of the moisture meter. Observation: the material was squeezed and it formed in my fist and when I let go, it crumbled.

9. Extracted mash from the ribbon mixer into mash tuns at 160 kilos per bin.

10. Once mash is in bins, we prepared to add Limonene to mash.

11 Added Limonene to the mash where liquid is just above the height of the mash in the tub approximately 75-85 gallons of limonene and

12. It sat for 24 hours.

### Example 2: Phase 2: Separation and Salting

1. Mash tanks were moved into place under the hoist systems using a forklift

2. Lids were removed from tanks/bins

3. Mash and Limonene was visually checked for temperature, color and texture.

Mash tanks were steaming and exceeded 90° F. Color was dark and shiny and texture was thick and smooth

4. Dewatering sacks containing Mash were hooked up to chain hoists and raised. Gravity allowed limonene to be separated from the Mash mixture and left to “drip” for a minimum of 30 minutes.
a. Alternative: dewatering has also been performed using a screw press and then centrifuged at 5000 RPM for 30 to 90 minutes.
b. Alternative: configuration at 500 RPM @ 1000-3000 G force.

5. Tanks/bins were connected with tubing/hoses that lead through “inline” filtration system. Filtration set up through tertiary filtration skid, was composed of six inline filters which include 50 micron to 10 micron down to absolute sub micron. This process is referred to as “pulls”.

6. Hoses/piping leading from filtration system was connected to cleaned and sanitized conical fermenter.

7. Vacuum pressure was applied to conical fermenter as to pull filtered alkaloid mixture (having kratom alkaloids, base, limonene water mixture) from tanks/bins through the inline filtration system and into the conical fermenter.

8. After all mixture is filtered and Mash bins were empty, the system was shut down and tanks were depressurized.

9. Liquid in conical fermenter was measured

10. Glacial acetic acid (GAA) was mixed with reverse osmosis water at a 1 to 9 ratio

11. 100 ml of 10% GAA solution was added for every gallon of limonene to tank. 10% GAA was made by obtaining pure GAA and diluted in reverse osmosis water. Spray ball was used to add the GAA to the mixture.

12. Alarm of 30 minutes was set to time. The process of glacial acetic acid solution settling to the bottom of conical fermenter “grabbing” alkaloids as it settles

13. Mash sacks were lowered back into bins/tanks and limonene was reapplied until all Mash was submerged and tanks are recovered with lids

14. Mash was left to “rest” for a minimum of 2 hours

15. When alarm on timer sounds, the mixture was run off the bottom of a conical fermenter through a valve to check the status of mixture

16. The mixture met the criteria which is measured by color, thickness, stickiness and a refractometer, the liquid mixture was sent to lab to be processes (Phase 3). The criteria of the refractive index is between 23-33. The refractive index was above 23. The color was and should be dark (amber to black onyx). The viscosity is high and the liquid is sticky. Phase separation occurs in the conical fermenter. The Limonene floated to the top and the GAA/alkaloid mixture, more dense liquid, went to the bottom of the conical fermenter. The two phases were visually seen with an emulsion layer between these phases. Using the valve at the bottom of the conical fermenter, the GAA/alkaloid mixture was drained out until reaching the emulsion layer.

17. Alternative: If criteria was met (refractive index was less than 22, color of liquid was light, viscosity was low or fluid is “thin”), the mixture was then put back into conical fermenter, steps 9-14 were repeated, and alarm timer was then reset for another 30 minutes

18. Process can be repeated no less than 2 times and no more than 4 for each application of glacial acetic acid solution into conical fermenter

All persons involved in the “pull” and “salting” are required to wear safety gear including ear plugs, protective glasses, chemical resistant gloves and apron and slip resistant shoes.

### Example 3: Phase 3 Crystallization

1. The mixture with a refractive index above 23, from Step 16 in Example 2, underwent Phase 3, crystallization, as follows.

2. Started a mitragynine seed—take a portion of the mitragynine seed from a previous batch

3. The GAA/alkaloid mixture with a refractive index above 23 (from Step 16 in Example 2) was filtered. Filtering removed any remaining D-limonene in the mixture. Filtering was done with a Buchner funnel and cotton balls. A filter was made with cotton balls and the mixture was poured over the cotton which removed limonene

4. The refractive index of the filtered mixture was measured using a refractometer.

5. A mixture with a refractive index above 25 was combined with seed mitragynine crystals and was put in a heated oven at 160° F. Crystallization occurred overnight—about 12 hours—and formed a crystalline solid or cake.

6. In cases when the refractive index is below 25, the mixture is combined with seed mitragynine crystals in 20 liter heated bath with sonication at a high frequencies using a sonicator (Model Vevor ultrasonic TH-100A; 600 watts; 40 kHz). This catalyzed the crystallization of the mitragynine crystals at. 40 kHz. Sonication formed crystalline solid or cake. Multiple sonication baths were run at the same time. This was performed between 30 minutes—3 hours to get crystallization. Sonication obtained higher returns secondary alkaloids, along with mitragynine.

7. The crystalline solid or cake was washed with reverse osmosis water using a filter and funnel. Water was poured over the crystalline solid or cake and the filter captured the crystals allowing the GAA to move through it, thereby separating the GAA and other contaminants from the crystals. This process was assisted with vacuum pressure.

8. Runoff and Wash liquid is collected. Base is added depending on the RI and pH to bring it into an appropriate pH range and sonicated for a longer period of time. This step is repeated 3 times.

9. The crystals were analyzed by a third party Murrey & Brown (Colorado USA) using HPLC. Test results show that the crystals have 70% mitragynine by weight.

10. Washed crystals were dehydrated into powder form using a dehydration oven at 150-170° F. for 12-18 hours.

11. After step 9, the dehydrated crystals were ground into a find powder using a hammermills. Final product had 70% mitragynine by weight.

### Example 4: Composition of Product

The product made from carrying out the methodology described herein, e.g., from Examples 1-3, were analyzed on multiple occasions by an independent laboratory (NN Analytics, San Diego California). The results are as follows:

TABLE 3
Composition Of Crystalline Product That Underwent Heating (Step 308 or 428)

| | | Method Group | | | | |
|---|---|---|---|---|---|---|
| Final | | Kratom | Method Name | | | |
| Disposition | | Alkaloids | WKI-03-0107 | | | |
| Method | | LOD | LOQ | Results | Results | Results |
| Group | Analyte / Property | (mg/g) | (mg/g) | (%) | (mg/g) | (mg/Unit) |
| Kratom | 7-hydroxymitragynine | N/A | N/A | ND | ND | N/A |
| Alkaloids | Mitraphyliline | N/A | N/A | ND | ND | N/A |
| | Isorhynchophylline | N/A | N/A | ND | ND | N/A |
| | Mitragynine | N/A | N/A | 91.88% | 918.8 | N/A |
| | Paynantheine | N/A | N/A | ND | ND | N/A |
| | Speciogynine | N/A | N/A | 2.63% | 26.3 | N/A |
| | Speciocilatine | N/A | N/A | 1.70% | 17.0 | N/A |
| | Total Alkaloids | | | 96.2% | 962.1 | N/A |

NOTES:
ND = Not Detected/ 1 μg/mL = 1 ppm / 1000 μg/mL = 1 mg/mL / 1% = 10 mg/g

TABLE 4
Composition Of Crystalline Product That Underwent Heating (Step 308 or 428)

| | | Method Group | | | | |
|---|---|---|---|---|---|---|
| Final | | Kratom | Method Name | | | |
| Disposition | | Alkaloids | WKI-03-0107 | | | |
| Method | | LOD | LOQ | Results | Results | Results |
| Group | Analyte / Property | (mg/g) | (mg/g) | (%) | (mg/g) | (mg/Unit) |
| Kratom | 7-hydroxymitragynine | N/A | N/A | ND | ND | N/A |
| Alkaloids | Mitraphyliline | N/A | N/A | ND | ND | N/A |
| | Isorhynchophylline | N/A | N/A | ND | ND | N/A |
| | Mitragynine | N/A | N/A | 89.23% | 892.3 | N/A |
| | Paynantheine | N/A | N/A | 2.31% | 23.1 | N/A |
| | Speciogynine | N/A | N/A | 1.65% | 16.5 | N/A |
| | Speciocilatine | N/A | N/A | 1.28% | 12.8 | N/A |
| | Total Alkaloids | | | 94.5% | 944.7 | N/A |

NOTES:
ND = Not Detected/ 1 μg/mL = 1 ppm / 1000 μg/mL = 1 mg/mL / 1% = 10 mg/g

The analysis shows that crystallization using heat results in a highly pure alkaloid composition. In Table 3, the total alkaloid in the final product is 96.2% wherein 91.88% is mitragynine and 4.33% are secondary alkaloids. Table 4 shows similar results with 94.5% total alkaloids with 89.23% mitragynine and 5.24% secondary alkaloids. Both sets of analysis show a highly pure product, well above 55%, performed in an industrial bulk processing facility.

TABLE 5
Composition Of Product That Underwent Sonication (Step 310 or Step 430)

| | | Method Group | | | | |
|---|---|---|---|---|---|---|
| Final | | Kratom | Method Name | | | |
| Disposition | | Alkaloids | WKI-03-0107 | | | |
| Method | | LOD | LOQ | Results | Results | Results |
| Group | Analyte / Property | (mg/g) | (mg/g) | (%) | (mg/g) | (mg/Unit) |
| Kratom | 7-hydroxymitragynine | N/A | N/A | ND | ND | N/A |
| Alkaloids | Mitraphyliline | N/A | N/A | ND | ND | N/A |
| | Isorhynchophylline | N/A | N/A | ND | ND | N/A |
| | Mitragynine | N/A | N/A | 80.87% | 808.7 | N/A |
| | Paynantheine | N/A | N/A | 2.48% | 24.8 | N/A |
| | Speciogynine | N/A | N/A | 2.65% | 26.5 | N/A |
| | Speciocilatine | N/A | N/A | 11.41% | 114.1 | N/A |
| | Total Alkaloids | | | 97.4% | 974.1 | N/A |

NOTES:
ND = Not Detected/ 1 μg/mL = 1 ppm / 1000 μg/mL = 1 mg/mL / 1% = 10 mg/g

The analysis from Table 5 shows that crystallization using sonication also results in a highly pure alkaloid composition. In Table 5, the total alkaloid in the final product is 97.4%. However, crystallization by sonication results in an increase of secondary alkaloids which are traditionally difficult to purify. In this case, the product has 80.87% mitragynine and 16.54% are secondary alkaloids with over 11.41% Speciocilatine, 2.65% Speciogynine and 2.48 Paynantheine. This is a surprising result. The method of crystallization, heating vs sonication, resulted in a different composition of secondary alkaloids. Secondary alkaloids are desired in certain circumstances such as for treating various conditions. For example, Speciocilatine, Speciogynine and/or Paynantheine can be used for pain management and/or to treat opioid addictions. All sets of analysis show a highly pure product, well above 55%, performed in an industrial bulk processing facility.

The terms about, approximately, substantially, and their equivalents may be understood to include their ordinary or customary meaning. In addition, if not defined throughout the specification for the specific usage, these terms can be generally understood to represent values about but not equal to a specified value. For example, 1%, 0.9%, 0.8%, 0.7%, 0.6%, 0.5%, 0.4%, 0.3%, 0.2%, 0.1%, 0.09% of a specified value.

The terms, comprise, include, and/or plural forms of each are open ended and include the listed items and can include additional items that are not listed. The phrase “And/or” is open ended and includes one or more of the listed items and combinations of the listed items.

The relevant teachings of all the references, patents and/or patent applications cited herein are incorporated herein by reference in their entirety. Citation of the above documents and studies is not intended as an admission that any of the foregoing is pertinent prior art. All statements as to the contents of these documents are based on the information available to the applicants and do not constitute any admission as to the correctness of the contents of these documents.

While this invention has been particularly shown and described with references to preferred embodiments thereof, it will be understood by those skilled in the art that various changes in form and details may be made therein without departing from the scope of the invention encompassed by the appended claims.

What is claimed is:

1) A method for purifying one or more chemical components from a starting plant material; the method comprises:
a) solubilizing the starting plant material in a solubilizer and a solvent, to thereby obtain a mixture having non-soluble plant material, one or more solubilized chemical components, and the solvent;
b) separating non-soluble plant material from the one or more solubilized chemical components and the solvent, to thereby obtain a mechanically separated solution;
c) salting the mechanically separated solution with a salting agent and separating the solvent from the one or more solubilized chemical components, to thereby obtain a salted solution having one or more chemical components, and the salting agent;
d) crystalizing the salted solution having one or more chemical components and the salting agent, to thereby obtain a crystalline solid having the one or more chemical components and the salting agent; and
e) separating the salting agent from the crystalline solid having the one or more chemical components to thereby obtain a purified crystalline solid having the one or more chemical components;
wherein the purified crystalline solid comprises an amount of the one or more chemical components ranging from about 55% w/w and about 100% w/w.

2) The method of claim 1, wherein the starting material has a weight ranging from about 100 kilograms to about 1000 kilograms.

3) The method of claim 1, further comprising dehydrating the purified crystalline solid having the one or more chemical components into a powder to obtain a dried product.

4) The method of claim 1, wherein the starting plant material comprises bark, root system, pollen, root stem, shoot system, stem, leaf, flower, fruit.

5) The method of claim 1, wherein after solubilizing the starting plant material in acid or base, the method further comprises adjusting a moisture content of the mixture having non-soluble plant material and one or more solubilized chemical components to a range from about 4% and about 70%.

6) The method of claim 1, wherein Step b) comprises separating the non-soluble plant material from one or more solubilized chemical components and the solvent, comprises gravity separation, force separation, centrifuge separation, or a combination thereof.

7) The method of claim 1, further comprising filtering the mechanically separated solution one or more times.

8) The method of claim 1, wherein Step c) further includes separating the solvent with phase separation to obtain the salted solution having one or more solubilized chemical components, and the salting agent.

9) The method of claim 8, wherein phase separation is performed with a conical fermenter.

10) The method of claim 1, wherein the one or more chemical components is present in an amount ranging from about 70% w/w and about 100% by weight.

11) The method of claim 1, wherein the one or more chemical components is present in an amount ranging from about 90% w/w and about 100% by weight.

12) A method for purifying one or more chemical components from a starting plant material; the method comprises:
a) solubilizing the starting plant material having a weight ranging from about 100 kilograms to about 1000 kilograms in an acid or base, and a solvent, wherein the solubilized plant material has a moisture content ranging between about 4% and about 70%, to thereby obtain a mixture having non-soluble plant material, one or more solubilized chemical components and the solvent;
b) mechanically separating the non-soluble plant material from the one or more solubilized chemical components and the solvent, to thereby obtain a mechanically separated solution;
c) filtering the mechanically separated solution to further remove non-soluble plant material one or more times to thereby obtain a filtered solution having the solubilized one or more chemical components and solvent;
d) salting the filtered solution with a salting agent and separating at least a portion of the solvent from the salted solution, to thereby obtain a salted solution having one or more chemical components, the salting agent, and a residual portion of the solvent;
e) measuring the refractive index, a pH or both of the salted solution, wherein the refractive index, the pH or both meet a criteria to thereby obtain a salted solution meeting the criteria;
f) separating the residual portion of the solvent from the salted solution meeting the criteria, to thereby obtain a filtered salted solution;
g) crystalizing the filtered salted solution, to thereby obtain a crystalline solid having the one or more chemical components and the salting agent;
h) separating the salting agent from the crystalline solid having the one or more chemical components to thereby obtain a purified crystalline solid having the one or more chemical components; and
i) dehydrating the purified crystalline solid having the one or more chemical components into a powder to obtain a dried product;
wherein the dried product comprises an amount of the one or more chemical components ranging from about 55% w/w to about 100 w/w.

13) The method of claim 12, wherein the starting plant material comprises bark, root system, pollen, root stem, shoot system, stem, leaf, flower, fruit.

14) The method of claim 12, wherein the starting plant material is derived from one or more of the following plant species: Acer saccharinum (Maple), Argemone Mexicana (Argemone-Prickly Poppy), Chionanthus virginicus (Fringe tree), Abies balsamea (L.)(Mill., Balsam Fir), Acacia senegal (Acacia), Achillea millefolium (Yarrow), Acorus calamus (Calamus root), Adiantum capillus-veneris (Maidenhair Fern), Aesculus hippocastanum (Horse Chestnut), Agaricus blazei (mushroom), Agathosma betulina (Buchu), Agave tequilana (Agave syrup), Agrimonia eupatoria L. (Agrimony), Agropyron repens (Couch grass), Alchemilla vulgaris (Lady's Mantle) Aletris farinosa (Unicorn Root—Colic Root), Aleurites moluccanus (Kukui Nut Oil), Allium sativum (Garlic Aloe vera, Aloe Vera Gel), Aloysia citriodora (Lemon Verbena), Alpinia officinarum (Galangal Root), Althaea officinalis L. (Marsh Mallow Root), Anacardium occidentale (Cashew Nut), Ananas comosus (Pineapple), Andrographis paniculata (Andrographis), Anethum graveolens (Dill), Angelica archangelica (Angelica root), Angelica sinensis (Dong Quai), anthriscus cerefolium (Chervil), Apium graveolens (Celery Seed), Aralia racemosa (Spikenard), Arctium lappa (Burdock root), Arctostaphylos uva-ursi (Uva Ursi), Argania spinosa (Argan Oil), Aristolochia spp (Snakeroot), Armoracia rusticana (Horseradish root), Arnica montana (Arnica), Artemisia absinthium L. (Wormwood), Artemisia dracunculus (Tarragon), Artemisia vulgaris (Mugwort), Arthrospira platensis (Spirulina), Asarum canadense (Wild Ginger), Asclepias syriaca or A. cornute (Milkweed), Asclepias tuberosa (Pleurisy Root), Aspalathus linearis (Rooibos), Asparagus spp (Asparagus), Astragalus membranaceus (Astragalus root), Atropa belladonna (Belladonna), Avena sativa L. (Oats), Azadirachta indica (Neem), Bacopa monnieri, Bacopa, Berberis vulgaris L., Barberry, Betula alba, Birch bark & leaf, Bixa orellana, Annatto Seed, Borago officinalis, Borage Seed Oil, Boswellia thurifera, Boswellia (Frankincense), Brassica napus, Canola oil rapeseed, Brassica nigra, Mustard, Brosimum alicastrum, Ramon Nut, Bupleurum chinense, Bupleurum, Butyrospermum parkii, Shea Nut Butter, Calendula officinalis, Calendula, Calophyllum inophyllum, Tamanu Oil, Camellia sinensis, Tea, Camellia sinensis, Oolong Tea, Cananga adorata, Ylang-Ylang, Cannabis sativa, Hemp Oil, Capsella bursa-pastoris, Shepherd's Purse, Capsicum minimum, Cayenne Pepper, Caralluma fimbriata, Caralluma, Carapa guianensis, Andiroba Oil, Carica papaya, Papaya, Carthamus tinctorius, Safflower Oil, Carum carvi, Caraway Seed, Cassia nomame, Cassia nomame, Caulophyllum thalictroides, Blue Cohosh Root, Ceanothus americanus, Red Root, Cedrus atlantica, Cedarwood, Atlas, Centaurea cyanus, Cornflowers, Centaurium erythraea, Centaury, Centella asiatica (L.), Gotu Kola, Ceratonia siliqua, Carob, Chamaelirium luteum, False Unicorn Root, Chelidonium majus, Celandine, Chenopodium ambrosioides, pazote, Chrysanthemum leucanthemum, Oxeye Daisy, Chrysanthemum morifolium, C. sinense, Chrysanthemum, Cichorium intybus, Chicory root, Cimicifuga racemosa, Black Cohosh root, Cinchona spp, Peruvian Bark, Cinnamomum camphora, Camphor, Cinnamomum zeylanicum, C. cassia, Cinnamon, Citrus aurantifolia, Lime Oil, Citrus aurantium subsp. amara or bigaradia, Neroli, Citrus bergamia, Bergamot Oil, Citrus brigarade, Petitgrain Oil, Citrus limonum, Lemon, Citrus paradisi, Grapefruit, Citrus sinensis, Citrus spp., Orange, Citrus×aurantium subsp. amara, Bitter Orange, Cnicus benedictus, Blessed Thistle, Coccinia grandis, Ivy gourd, Cocos nucifera, Coconut oil, Codonopsis pilosula, Codonopsis, Coffea arabica, Coffee, Cola acuminata, Kola Nut, Commiphora myrrha, Myrrh, Convolvulus jalapa, Jalap, Copaifera Officinalis, Copaiba Balsam, Coptis spp, Coptis, Coriandrum sativum, Cilantro, Coriandrum sativum, Coriander, Corydalis spp, Corydalis, Corylus avellana, Hazelnut Oil, Crataegus monogyna, Hawthorn Berry, Crocus sativus, Saffron, Cucurbita pepo, Pumpkin Seed, Cuminum cyminum, Cumin, Cupressus sempervirens, Cypress Oil, Curcuma longa L., Turmeric, Cyclopia spp, HoneyBush, Cymopogon citratus, C. flexuosus, Lemongrass, Cynara scolymus, Artichoke, Globe, Datura spp, Datura, Daucus carota L., Carrot, Wild, Digitalis purpurea, foxglove digitalis, Dioscorea villosa, Wild Yam Root, Echinacea angustifolia, Echinacea, Elettaria cardamomum, Cardamom, Eleutherococcus senticosus, Eleuthero Root, Epilobium angustifolium, Fireweed, Epimedium grandiflorum, Epimedium, Equisetum arvense, Horsetail, Erythroxylum catuaba, Catuaba, Erythroxylum coca, Coca, Eschscholzia californica, California Poppy, Eucalyptus globulus, Eucalyptus, Eugenia jambolana, Jambul, Eupatorium perfoliatum, Boneset, Eupatorium purpureum, Joe-pye weed, Euphrasia, various species, Eyebright, Euterpe oleracea, Acai Berry, Ferula asafoetida, Asafoetida root, Filipendula ulmaria, Meadowsweet, Foeniculum vulgare, Fennel Seed, Forsythia suspensa, Forsythia Fruit, Fucus vesiculosis, Kelp/Bladderwrack, Fumaria officinalis, Fumitory, Galega officinalis, Goat's Rue, Galium aparine, Cleavers, Galium odoratum, Sweet Woodruff, Ganoderma lucidum, Reishi Mushroom, Garcinia cambogia, Garcinia Fruit, Gaultheria procumbens, Wintergreen Oil, Gentiana lutea, Gentian, Ginkgo biloba L., Ginkgo biloba, Glechoma hederacea L, Ground Ivy, Glycyrrhiza glabra, Licorice Root, Grifola frondosa, Maitake mushroom, Grindelia spp, Grindelia, Gymnema sylvestre, Gurmar, Gynostemma pentaphyllum, Jiaogulan, Hamamelis virginiana, Witch Hazel, Harpagophytum procumbens, Devil's Claw, Helianthemum canadense (L.) Michx., Rock Rose, Helianthus annuus, Sunflower, Helichrysum angustifolium, Immortelle Oil, Hepatica nobilis var. obtusa, Hepatica Liver-Leaf, Hibiscus sabdariffa, Hibiscus, Hippophae rhamnoides, Sea Buckthorn Oil, Hoodia gordonii, Hoodia, Hordeum vulgare, Barley Grass, Humulus Lupulus, Hops, Hyacinthus nonscriptus, Wild Hyacinth, Hydrastis canadensis, Goldenseal, Hyoscyamus niger, Henbane, Hypericum perforatum, St. John's Wort, Hyssopus officinalis, Hyssop, Ilex paraguariensis, Yerba Mate, Impatiens capensis, Jewel Weed, Inonotus obliquus, Chaga Mushroom, Inula helenium, Elecampane, Iris germanica, Orris Root, Jasminum officinale, Jasmine Flower Oil, Juglans cinerea, Butternut, Juglans nigra, Black Walnut, Juniperus communis, Juniper Berries, Lactuca virosa, Wild lettuce, Larrea tridentata, Chaparral, Laurus nobilis, Bay Laurel, Lavandula spp, Lavender, Lawsonia inermis, Henna, Leonurus caridica L., Motherwort, Lepidium peruvianum, Maca root, Levisticum officinale, Lovage, Liatris odoratissima, Deer's Tongue, Linum usitatissimum L., Flax seed, Lippia graveolens, Mexican Oregano, Lobelia inflata, Lobelia, Lonicera periclymenum, Honeysuckle, Lycium barbarum, Goji Berries, Lycopodium clavatum, Club Moss, Lycopus spp., Bugleweed, Magnolia glauca, Magnolia, Mahonia aquifolium, Oregon Grape root, Malus domestica, Apple, Maranta arundinacea, Arrowroot powder, Marrubium vulgare L., Horehound, Matricaria recutita, Chamomile, Medicago sativa L., Alfalfa, Melaleuca alternifolia, Tea Tree oil, Melaleuca leucadendron, M. leucadendra, Cajeput Oil, Melissa officinalis, Lemon Balm, Mentha piperita, Peppermint, Mentha pulegium, PennyRoyal, Mentha spicata, Spearmint, Menyanthes trifoliata, Bogbean, Momordica charantia, Bitter Melon, Monarda didyma, Bee Balm, Wild Bergamot, Morinda citrifolia, Noni, Myrica cerifera, Bayberry, Myristica fragrans, Nutmeg, Myroxylon pereirae, Balsam of Peru, Myrtus communis, Myrtle, Nardostachys jatamansi, Nard Oil, Nasturtium officinale, Watercress, Nelumbo nucifera, Lotus, Nepeta cataria, Catnip, Nigella sativa, Black Seed oil, Ocimum basilicum, Basil, Ocimum sanctum, Holy Basil, Oenothera biennis, Evening Primrose Oil, Olea europea, Olive Oil, Opuntia ficus-indica, Prickly Pear, Origanum majorana, Marjoram, Sweet, Origanum vulgare, Oregano, Paeonia albiflora, White Peony root, Panax ginseng, Ginseng root, Panax quinquefolius, Ginseng Root, American, Papaver rhoeas, Red Poppy Flowers, Passiflora incarnata, Passion Flower, Paullinia cupana, Guarana, Pausinystalia johimbe, Yohimbe, Pelargonium graveolens, Geranium, Persea americana, Avocado, Petasites vulgaris, Butterbur, Petroselinum crispum, Parsley, Peumus boldus Molina, Boldo, Pfaffia paniculata, Suma Root, Phaseolus vulgaris, White Kidney Bean, Phyllanthus emblica, Amalaki, amla, Phyllanthus niruri, Chanca piedra, Phytolacca americana, Poke Root, Picea mariana, Spruce Oil, Pimenta officinalis, Allspice, Pimenta racemosa, Bay Rum Oil, Pimpinella anisum, Anise Seed, Pinus sylvestris, Pine, Piper cubeba, Cubeb, Piper longum, Long Pepper, Pippali, Piper methysticum, Kava-Kava, Piper nigrum, Black Pepper, Piscidia piscipula, Jamaican Dogwood, Plantago major, lanceolata L., Plantain, Plantago psyllium, Plantago ovata, Psyllium, Plectranthus barbatus, Coleus Forskohlii, Podophyllum peltatum, MayApple, Pogostemon patchouli, Patchouli, Polygonatum biflorum, Solomon's Seal, Polygonum multiflorum, Fo-Ti Root, Populus spp, Balm of Gilead, Prunella vulgaris L., Self-Heal, Prunus africanum, Pygeum, Prunus armeniaca, Apricot Oil, Prunus dulcis, Almond Oil, Prunus serotina, Wild Cherry Bark, Ptychopetalum olacoides, Muira puama, Pueraria lobata, Kudzu, Pulsatilla nuttalliana, Pasque Flower, Punica granatum, Pomegranate, Quassia amara, Quassia, Quercus robur, Oak Bark, Ravensara aromatica, Ravensara Oil, Rehmannia glutinosa, Rehmannia root, Rhamnus frangula, Alder Buckthorn, Rhamnus purshiana DC, Cascara Sagrada, Rheum palmatum, Turkey Rhubarb, Rhodiola rosea, Rhodiola, Ricinus communis, Castor Oil, Rosa spp, Rose, Rosmarinus officinalis, Rosemary, Rubus idaeus, Raspberry Leaf, Rumex acetosella var. vulgaris, Sheep Sorrel, Rumex crispus, Yellow Dock, Ruscus aculeatus, Butcher's Broom, Ruta graveolens L., Rue, Salix Alba, White Willow Bark, Salvia officinalis, Sage, Salvia sclarea, Clary Sage, Sambucus nigra, Elderberry, Sanguinaria canadensis L, Bloodroot, Sanguisorba minor, Salad Burnet, Santalum album, Sandalwood Oil, Saponaria officinalis, Soapwort, Sassafras albidum, Sassafras root, Satureja montana, Savory, Winter, Summer, Sceletium tortuosum, Sceletium, Schisandra chinensis, Schisandra, Scrophularia nodosa, Figwort, Scutellaria lateriflora, Skullcap, Senna alexandrina Mill., Senna, Serenoa serrulata, Saw Palmetto, Sesamum indicum, Sesame, Silybum marianum L., Milk Thistle Seed, Simmondsia chinensis, Jojoba Oil, Smallanthus sonchifolius, Yacon Root, Smilax sarsaparilla, Sarsaparilla Root, Solidago virgaurea, Goldenrod, Spilanthes acmella, Spilanthes, Stachys officinalis, Wood Betony, Stellaria media, Chickweed, Stevia rebaudiana (Bertoni), Stevia, Styrax benzoin, Benzoin Resin, Symphytum officinale, Comfrey Leaf and root, Syzygium aromaticum, Clove Oil, Tabebuia spp, Pau d'arco, Tanacetum balsamita L., Costmary, Tanacetum parthenium, Feverfew, Tanacetum vulgare, Tansy, Taraxacum officinale, Dandelion Root, Terminalia arjuna, Arjuna, Theobroma cacao L., Cacao, Thuja occidentalis, Thuja oil, Thymus vulgaris, Thyme, Tilia spp, Linden, Trachyspermum ammi, Ajwain Seed, Tribulus terrestris, Tribulus terrestris L, Trifolium pratense, Red Clover, Trigonella foenum-graecum, Fenugreek, Trillium erectum, Beth Root, Turnera diffusa, Damiana, Tussilago farfara, Colt's Foot, Ulmus fulva, Elm, Slippery, Uncaria tomentosa, Cat's Claw, Urtica dioica, Stinging Nettle, Vaccinium macrocarpon, Cranberry, Vaccinium myrtillus, Bilberry, Vaccinium spp, Blueberries, Valeriana officinalis, Valerian root, Vanilla planifolia, Vanilla, Verbascum spp., Mullein, Verbena hastata, V. officinalis, Vervain, Veronica officinalis, Speedwell, Vetiveria zizanoides, Vetiver Oil, Viburnum opulus, Cramp Bark, Vinca minor, Periwinkle, Viola spp, Violet Leaf, Viscum coloratum, V. album, Mistletoe, Vitex agnus-castus, Chaste Tree, Vitis vinifera, Grapes, Withania somnifera, Ashwagandha root, Yucca spp, Yucca Root, Zanthoxylum spp, Prickly Ash, Zea mays, Corn Silk, Zingiber officinale, or Ginger Root.

15) The method of claim 12, wherein the base comprises calcium hydroxide, sodium carbonate, sodium hydroxide, potassium hydroxide, potassium carbonate or a combination thereof.

16) The method of claim 12, wherein the acid comprises glacial acetic acid, hydrochloric acid, sulfuric acid, nitric acid, sulfuric acid, carbonic acid, hydrofluoric acid, citric acid, phosphoric acid, ascorbic acid, or a combination thereof.

17) The method of claim 12, wherein the solvent comprises limonene, p-cymene, pinene, cyclopentyl ethyl ether, ethyl lactate, 2-methyloxolaine, glycerol, Isopopanol, n-propylacetate, i-propylacetate, 1-butanol, 2-butanol, sulpholane, anisole, ethylene, propylene carbonate, dimethyl carbonate, tert amyl methyl ether, methyl laurate, 2-methyltetrahydrofuran, 2,2,5,5-tetramethyloxolane, cetyltrimethylammonium chloride, dimethyldodecylamine oxide, sodium dodecyl sulfate, dimethoxyethane, methylene chloride, polyethylene glycol, ethanol, methanol, petroleum ether, n-hexane, chloroform, methyl chloride, ethyl acetate, hexane, toluene, naptha, xylene, or combinations thereof.

18) The method of claim 12, wherein Step b) comprises mechanically separating non-soluble plant material from the one or more solubilized chemical components and solvent comprises gravity separation, force separation, centrifuge separation, or a combination thereof.

19) The method of claim 12, wherein the Step c) comprises filtering the mechanically separated solution two or more times.

20) The method of claim 11, wherein the salting agent of Step d) comprises glacial acetic acid, hydrochloric acid, sulfuric acid, nitric acid, sulfuric acid, hydrofluoric acid, carbonic acid, citric acid, phosphoric acid, ascorbic acid, calcium hydroxide, sodium carbonate, sodium hydroxide, potassium hydroxide, potassium carbonate or a combination thereof.

21) The method of claim 12, wherein Step d) further includes separating the portion of the solvent with phase separation to obtain the salted solution having one or more chemical components, the salting agent, and a residual portion of the solvent.

22) The method of claim 21, wherein phase separation is performed in a conical fermenter.

23) The method of claim 12, wherein the criteria for the refractive index comprises a refractive index of greater than about 22.

24) The method of claim 11, wherein the criteria for the pH comprises a pH greater than about 4.0.

25) The method of claim 12, wherein crystalizing the mixture having one or more chemical components comprising using seed crystals.

26) The method of claim 25, wherein step g) comprises heating, sonicating or both.

27) The method of claim 12, wherein Step h) comprises performing one or more washes.

28) The method of claim 12, wherein Step i) comprises subjecting the crystalline solid of Step h) to a dehydrator at a temperature for a time period.

29) The method of claim 12, wherein the dried product comprises an amount of the one or more chemical components of at least about 55%, 60%, 65%, 70%, 75%, 80%, 85%, 90%, or 95% w/w.

30) The method of claim 29, wherein the one or more chemical components comprises one or more alkaloids.

31) A purified crystalline solid comprising an amount of the one or more plant derived chemical components ranging from about 55% and about 100%, wherein the one or more plant derived chemical components is derived from a starting plant material having a weight ranging from about 100 kilograms to about 1000 kilograms.

32) The purified crystalline solid of claim 31 wherein the amount of the one or more plant derived chemical components is about 55%, 60%, 65%, 70%, 75%, 80%, 85%, 90%, or 95% w/w.

33) The purified crystalline solid of claim 31, wherein the one or more plant derived chemical components comprises mitragynine, speciocilatine, speciogynine, paynantheine, ajmalicine, akuammigine, ciliaphylline, corynantheidine, corynoxeine, corynoxine A, corynoxine B, epicatechin, 9-hydroxycorynantheidine, 7-hydroxymitragynine, isomitraphylline, isomitrafoline, isopteropodine, isorhynchophylline, isospeciofoline, mitraciliatine, mitragynine n-oxide, mitragynine oxindole B, mitragynine pseudoindoxyl, mitrafoline, Mitraphylline, mitraversine, thrnchophylline, speciociliatine n-oxide, speciofoline, speciophylline, stipulation, tetrahydroalstonine or their stereoisomers.

34) A purified crystalline solid comprising an amount of a primary alkaloid ranging from about 70% to about 100% and the amount of one or more secondary alkaloids ranging from about 1% to about 30%.

35) The purified crystalline solid of claim 34, wherein the amount of the primary alkaloid is about 70%, 75%, 80%, 85%, 90%, or 95% w/w and the amount of the one or more secondary alkaloids is about 1%, 2%, 3%, 4%, 5%, 6%, 7%, 8%, 9%, 10%, 15%, 20%, 25%, or 30% w/w.

36) The purified crystalline solid of claim 34, wherein the primary alkaloid comprises mitragynine or an isomer thereof.

37) The purified crystalline solid of claim 34, wherein the one or more secondary alkaloids comprises speciocilatine, speciogynine, paynantheine, ajmalicine, akuammigine, ciliaphylline, corynantheidine, corynoxeine, corynoxine A, corynoxine B, epicatechin, 9-hydroxycorynantheidine, 7-hydroxymitragynine, isomitraphylline, isomitrafoline, isopteropodine, isorhynchophylline, isospeciofoline, mitraciliatine, mitragynine n-oxide, mitragynine oxindole B, mitragynine pseudoindoxyl, mitrafoline, Mitraphylline, mitraversine, thrnchophylline, speciociliatine n-oxide, speciofoline, speciophylline, stipulation, tetrahydroalstonine or their stereoisomers.

38) The purified crystalline solid of claim 34, wherein the purified crystalline solid is crystalized with sonication.

39) A purified crystalline solid having an amount of the one or more chemical components ranging from about 55% and about 100%, wherein the purified crystalline solid is made from a method comprising the steps of:
a) solubilizing the starting plant material having a weight ranging from about 100 kilograms to about 1000 kilograms in a solubilizer with a solvent, to thereby obtain a mixture having non-soluble plant material and one or more solubilized chemical components, and the solvent;
b) separating non-soluble plant material from the one or more solubilized chemical components, and the solvent, to thereby obtain a mechanically separated solution;
c) salting the mechanically separated solution with a salting agent and separating the solvent from the one or more solubilized chemical components, to thereby obtain a salted solution having one or more chemical components, and the salting agent;
d) crystalizing the salted solution having one or more chemical components, and the salting agent, to thereby obtain a crystalline solid having the one or more chemical components, and the salting agent; and
e) separating the salting agent from the crystalline solid having the one or more chemical components to thereby obtain a purified crystalline solid having the one or more chemical components;
wherein the purified crystalline solid comprises an amount of the one or more chemical components ranging from about 55% w/w and about 100% w/w.
